(19) United States
(12) Patent Application Publication
(10) Pub. No.: US 2025/0171388 A1
(43) Pub. Date: May 29, 2025

(54) Process For the Production of Methanol and Hydrogen from Methane Using a Liquid Reactant

(71) Applicants: TOTALENERGIES ONETECH, Courbevoie (FR); SULZER MANAGEMENT AG, Winterthur (CH)

(72) Inventors: Gleb VERYASOV, Seneffe (BE); Nikolai NESTERENKO, Winterthur (CH); Zhongyi DING, Katy, TX (US); Mircea-Gheorghe CRETOIU, Sugar Land, TX (US); Miguel Alexandre FIGUEIREDO SANTOS, Sugar Land, TX (US); Jingsong ZHOU, Houston, TX (US)

(21) Appl. No.: 18/994,151

(22) PCT Filed: Jul. 24, 2023
(86) PCT No.: PCT/EP2023/070456

§ 371 Date: Jan. 14, 2025

(30) Foreign Application Priority Data
Jul. 25, 2022 (EP) 22315166.3
Jul. 25, 2022 (EP) 22315167.1
Jul. 25, 2022 (EP) 22315168.9

(51) Int. Cl. C07C 29/124; C07C 17/06; C07C 41/01; C25B 1/24; C25B 15/08

(52) U.S. Cl. CPC C07C 29/124; C07C 17/06; C07C 41/01; C25B 1/24; C25B 15/081

(57) ABSTRACT

The disclosure provides a process for producing methanol and hydrogen from methane. The process of the disclosure comprises the steps of: •a) providing a gaseous feed stream comprising methane: •b) reacting said gaseous feed stream with at least one halogen reactant •under reaction conditions effective to produce an effluent stream comprising methyl halide, hydrogen halide •optionally poly halogenated alkanes •and optionally unreacted methane: •c) recovering said an effluent stream •d) reacting the recovered effluent stream with water and at least one organic base under reaction conditions effective to produce an aqueous solution of hydrogen halide •and a methanol stream comprising methanol (MeOH) and dimethyl ether (DME) and/or optionally unreacted methane, and, c) decomposing by means of electrolysis said aqueous solution of hydrogen halide under conditions effective to produce a gaseous hydrogen stream and a stream comprising halogen reactant.

### TECHNICAL FIELD

The present disclosure relates to a process and a system for producing methanol. In particular, the present disclosure relates to a process and system for the production of methanol from a gas stream comprising or consisting of methane, thereby producing hydrogen and dimethyl ether (DME). The present process is, in particular, remarkable in that it utilizes water as a liquid reactant in the conversion process of methane into methanol, and in that it uses electrolysis to produce hydrogen gas.

### BACKGROUND

Methanol is a key starting material for the production of base chemicals such as formaldehyde, acetic acid, hydrocarbons, olefins, biodiesel, gasoline, and gasoline additives such as methyl tert-butyl ether. Methanol can also be used as transportation fuel, as a fuel cell hydrogen carrier, as well as in wastewater treatment, or in electricity production. It is then an excellent fuel and a key starting material for important industrial reactions. In the recent years, also, methanol is suggested as an alternative to chemical energy carriers.

Conventionally, methanol (CH₃OH or MeOH) is produced on an industrial scale from the synthesis gas “syngas,” which is a combination of varying amounts of H₂, CO, and CO₂ frequently derived from gasified coal or natural gas. Catalytic conversion of hydrogen (H₂) and carbon monoxide (CO) from coal-derived syngas into methanol involves the following reactions:

CO+2H₂→CH₃OH (I)

CO₂+3H₂→CH₃OH+H₂O (II)

CO+H₂O→CO₂+H₂ (III)

Catalyst systems used for methanol synthesis are typically mixtures of copper, zinc oxide, alumina, and magnesia.

All three reactions given above are highly exothermic. Especially, to favor the formation of methanol, high pressures and/or temperatures are needed to shift the equilibrium towards methanol.

Even more, the production of the syngas often involves the reforming of natural gas, which itself is very energy-consuming as the reaction with water is endothermic and is typically performed at high temperatures. A typical example of a cracking process is the nickel-catalyzed reaction of methane and steam in a 1 to 3 ratio, at 840° C. Thus, produced syngas will have a composition of 3 moles H₂ to every mole CO. Alternatively, the reaction of partial oxidation of methane with oxygen could be carried out to produce carbon monoxide. It could be combined with steam reforming in the same reaction zone to avoid the necessity for providing additional energy input for the reaction. However, partial oxidation (which is sometimes called autothermal reforming) would require the use of pure oxygen, i.e., such a plant would need an air separation unit—a highly capital-intensive and energy-consuming device for the production of pure oxygen from the air. In view of the above, the overall process of making methanol from syngas is complex, energetically consuming, and capital intensive.

Hence, in view of the above, there is a need in the art to provide improved processes for producing methanol.

There is a demand in the art for providing processes for producing methanol having improved efficiencies. There is in particular a demand in the art for methanol production processes that can operate under milder production process conditions, avoiding working at highly elevated temperatures and/or pressures that require high amounts of energy to maintain these temperatures and/or pressures.

There is also a demand in the art for methanol production processes that involve lower CO/CO₂ emissions.

There is a further demand in the art for methanol production processes allowing to produce methanol via a different strategy, especially not starting from a syngas route.

The document WO 2008/143940 concerns a continuous process for converting hydrocarbon feedstocks into higher-value products using molecular halogen to active C—H bonds in the feedstock and electrolysis to convert the hydrogen halide formed in the process back into molecular halogen. When methanol is formed, the formation of methanol is made by hydrolysis. The electrolytic recovery of halogen is performed from NaBr.

US 2002/0198416 relates to the formation of alcohol and/or ether from alkane using bromine and methane. Methanol is formed thanks to a metal oxide catalyst.

It is, therefore, an object of the present disclosure to provide a process for producing methanol and/or methanol in combination with dimethyl ether, which allows for fulfilling at least some of the above-indicated demands. For example, there is a need for a process for producing methanol, DME, and hydrogen from methane, with high selectivity to DME.

### SUMMARY

In accordance with the present disclosure an improved process for producing from methane methanol and hydrogen gas, and/or methanol, dimethyl ether, and hydrogen gas, is provided.

In a first aspect, the present disclosure thereto provides a process for producing methanol (MeOH) and hydrogen (H₂) from methane, comprising the steps of:
a) providing a gaseous feed stream comprising methane;
b) reacting said gaseous feed stream with at least one halogen reactant (X₂), under reaction conditions effective to produce an effluent stream comprising methyl halide (MeX), hydrogen halide (HX), optionally polyhalogenated alkanes, and optionally unreacted methane;
c) recovering an effluent stream comprising methyl halide (MeX), hydrogen halide (HX), optionally polyhalogenated alkanes, and optionally unreacted methane;
d) reacting the effluent stream obtained in step c) with water and at least one catalyst, under reaction conditions effective to produce:
   an aqueous solution of hydrogen halide (HX_{(aq)}), and
   a methanol stream comprising methanol (MeOH), and optionally dimethyl ether (DME) and/or optionally unreacted methane, and,
e) decomposing by means of electrolysis said aqueous solution of hydrogen halide (HX(aq)) under conditions effective to produce a gaseous hydrogen (H₂) stream and a stream comprising halogen reactant (X₂).

More particularly, the present disclosure thereto provides a process for producing methanol (MeOH), dimethyl ether (DME), and hydrogen (H₂) from methane, comprising the steps of:
a) providing a gaseous feed stream comprising methane;
b) reacting said gaseous feed stream with at least one halogen reactant (X₂), under reaction conditions effective to produce an effluent stream comprising methyl halide (MeX), hydrogen halide (HX), optionally polyhalogenated alkanes, and optionally unreacted methane;
c) recovering an effluent stream comprising methyl halide (MeX), hydrogen halide (HX), optionally polyhalogenated alkanes, and optionally unreacted methane;
d) reacting the effluent stream obtained in step c) with water and an organic base, under reaction conditions effective to produce:
   an aqueous solution of hydrogen halide (HX_{(aq)}), and
   a methanol stream comprising methanol (MeOH) and dimethyl ether (DME) and optionally unreacted methane, and,
e) decomposing by means of electrolysis said aqueous solution of hydrogen halide (HX(aq)) under conditions effective to produce a gaseous hydrogen (H₂) stream and a stream comprising halogen reactant (X₂).

In certain embodiments, the process comprises the step of separating said polyhalogenated alkanes formed in step b) from said effluent stream, preferably prior to reacting the effluent stream with water and at least one catalyst.

In certain embodiments, the process comprises the steps of:
a) providing a gaseous feed stream comprising methane;
b) reacting said gaseous feed stream with at least one halogen reactant (X₂), under reaction conditions effective to produce an effluent stream comprising methyl halide (MeX), hydrogen halide (HX), optionally polyhalogenated alkanes, and optionally unreacted methane;
c) separating from the effluent stream obtained in step b):
   c1) a mono-halide stream, comprising methyl halide (MeX) and hydrogen halide (HX), and optionally comprising unreacted methane; and,
   c2) a polyhalogenated alkanes stream,
d) reacting the mono-halide stream separated in step c1), with water and at least one catalyst, under reaction conditions effective to produce:
   d1) an aqueous solution of hydrogen halide (HX_{(aq)}), and
   d2) a methanol stream comprising methanol (MeOH), and optionally dimethyl ether (DME), and/or optionally unreacted methane,
e) decomposing by means of electrolysis said aqueous solution of hydrogen halide (HX_{(aq)}) produced in step d1) under conditions effective to produce a gaseous hydrogen (H₂) stream and a stream comprising halogen reactant (X₂); and,
f) optionally, recovering methanol from said methanol (MeOH) stream.

For example, the process comprises the steps of:
a) providing a gaseous feed stream comprising methane;
b) reacting said gaseous feed stream with at least one halogen reactant (X₂), under reaction conditions effective to produce an effluent stream comprising methyl halide (MeX), hydrogen halide (HX), optionally polyhalogenated alkanes, and optionally unreacted methane;
c) separating from the effluent stream obtained in step b):
   c1) a mono-halide stream, comprising methyl halide (MeX) and hydrogen halide (HX), and optionally comprising unreacted methane; and,
   c2) a polyhalogenated alkanes stream,
d) reacting the mono-halide stream separated in step c1), with water and an organic base, under reaction conditions effective to produce:
   d1) an aqueous solution of hydrogen halide (HX_{(aq)}), and
   d2) a methanol stream comprising methanol (MeOH) and dimethyl ether (DME), and optionally unreacted methane,
e) decomposing by means of electrolysis said aqueous solution of hydrogen halide (HX_{(aq)}) produced in step d1) under conditions effective to produce a gaseous hydrogen (H₂) stream and a stream comprising halogen reactant (X₂); and,
f) optionally, recovering methanol from said methanol (MeOH) stream and/or recovering dimethyl ether (DME) from said methanol (MeOH) stream.

In certain embodiments, said halogen reactant is selected from the group consisting of bromine (Br₂), chlorine (Cl₂), fluorine (F₂), iodine (I₂), and astatine (At₂). With preference, said halogen reactant is bromine (Br₂).

In certain embodiments, the at least one catalyst is a base, preferably an organic base. In certain embodiments, the at least one catalyst is an N-containing polymer, and preferably a polymer comprising at least one amino group, pyridine ring, pyridazine ring, pyrimidine ring, pyrazine ring, triazine ring, pyrazole ring, imidazole ring, oxazole ring, isoxazole ring, thiazole ring, isothiazole ring, triazole ring, oxadiazole ring, or any combinations thereof. With preference, said N-containing polymer is a polymer comprising at least one pyridine ring. In certain embodiments, said polyhalogenated alkanes comprise polyhalogenated methane and optionally polyhalogenated C₂₊ alkanes.

In certain embodiments, C₂₊ alkyl monohalides are formed in step b).

In certain embodiments, the process comprises the step of separating C₂₊ alkyl monohalides formed in step b) of the process from said effluent stream, and preferably prior to separating the mono-halide stream comprising methyl halide (MeX) and hydrogen halide (HX).

In certain embodiments, the gaseous feed stream comprises at least 75.0 vol % methane, preferably at least 80.0 vol % methane, preferably at least 85.0 vol % methane, preferably at least 90.0 vol % methane, preferably at least 95.0 vol % methane, preferably at least 98.0 vol % methane, preferably at least 99.0 vol % methane, preferably at least 99.9 vol % methane, based on the total volume of the gaseous feed stream.

In certain embodiments, the gaseous feed stream applied in step a) of the process is substantially free of sulphur species, e.g. it contains less than 1.0 mol %, or less than 0.5 mol %, or less than 0.1 mol %, or less than 0.01 mol %, or less than 0.001 mol %, or less than 0.0001 mol %, or less than 0.00001 mol % of sulphur species.

In certain embodiments, the process further comprises the step of returning the halogen reactant (X₂) obtained in step e), to step b) of the process.

In certain embodiments, the methanol stream comprising methanol (MeOH), and optionally dimethyl ether (DME), obtained in step d2) is separated from the aqueous solution of hydrogen halide by means of distillation, and preferably comprises, at least 75.0 mol %, such as least 80.0 mol %, or at least 85.0 mol %, or at least 95.0 mol % of methanol and optionally dimethyl ether in combination, based on the total amount of the methanol stream.

In certain embodiments, the gaseous hydrogen (H₂) stream generated in step e) comprises at least 90.0 mol %, such as at least 95.0 mol %, or at least 99.0 mol %, or at least 99.5 mol %, or at least 99.9 mol % hydrogen.

In a second aspect, the present disclosure thereto provides a system for producing methanol (MeOH) and hydrogen (H₂) from methane, comprising;
a halogenation reactor, configured to react a gaseous feed stream comprising methane with at least one halogen reactant (X₂) into an effluent stream comprising methyl halide (MeX), and hydrogen halide (HX), and optionally unreacted methane,
optionally, a polyhalogenated alkane removal unit, configured to receive an effluent stream from said halogenation reactor and configured to separate from said effluent stream (i) a polyhalogenated alkane stream, comprising polyhalogenated alkanes; and, (ii) a mono-halide stream, comprising methyl halide (MeX) and hydrogen halide (HX), and optionally comprising unreacted methane; and,
a hydrolysis reactor, which is fluidly connected to said halogenation reactor and configured to receive an effluent stream from said halogenation reactor; or, which is fluidly connected to said polyhalogenated alkane removal unit and configured to receive a mono-halide stream from said polyhalogenated alkane removal unit; and, wherein said hydrolysis reactor is configured to react the effluent stream or the mono-halide stream, with water and at least one catalyst, under reaction conditions effective to produce (i) an aqueous solution of hydrogen halide (HX_{(aq)}), and (ii) a stream comprising methanol (MeOH) and optionally dimethyl ether (DME) and/or optionally unreacted methane; and
an electrolysis unit comprising at least one electrolysis cell and a power source for supplying current to said electrolysis cell, which is fluidly connected to said hydrolysis reactor, and configured to receive an aqueous solution of hydrogen halide (HX_{(aq)}) and to decompose said aqueous solution of hydrogen halide (HX_{(aq)}) into a gaseous hydrogen (H₂) stream and a stream comprising halogen reactant (X₂);
a feed stream supply system for supplying a gaseous feed stream comprising methane to said halogenation reactor;
a halogen supply system for supplying a halogen reactant (X₂) to said halogenation reactor;
an effluent recovery system, configured to recover an effluent stream from said halogenation reactor, and for feeding said recovered effluent stream to said optional polyhalogenated alkane removal unit, or to said hydrolysis reactor;
a methanol recovery system, configured to recover a stream comprising methanol (MeOH), and optionally dimethyl ether (DME) and/or optionally unreacted methane from said hydrolysis reactor;
a hydrogen recovery system, configured to recover a gaseous hydrogen (H₂) stream from said electrolysis unit;
a halogen recovery system, configured to recover halogen reactant (X₂) from said electrolysis unit;
a hydrogen halide transfer system, configured to recover an aqueous solution of hydrogen halide (HX_{(aq)}) from said hydrolysis reactor, and to supply said recovered aqueous solution of hydrogen halide (HX_{(aq)}) to the electrolysis unit;
optionally, a mono-halide transfer system, configured to recover a mono-halide stream from said polyhalogenated alkane removal unit, and to supply said mono-halide stream to the hydrolysis reactor,
optionally, a water supply line, for supplying water, preferably water and at least one catalyst, to said hydrolysis reactor;
optionally, a C₂₊ alkyl monohalide removal unit, configured to receive effluent stream from said halogenation reactor and to remove C₂₊ alkyl monohalides from said effluent stream.

In particular, the disclosure also provides a system for producing methanol (MeOH), dimethyl ether (DME) and hydrogen (H₂) from methane, comprising;
a halogenation reactor, configured to react a gaseous feed stream comprising methane with at least one halogen reactant (X₂) into an effluent stream comprising methyl halide (MeX), and hydrogen halide (HX), and optionally unreacted methane; and,
a hydrolysis reactor, which is fluidly connected to said halogenation reactor and configured to receive an effluent stream from said halogenation reactor; and, wherein said hydrolysis reactor is configured to react the effluent stream with water and an organic base, under reaction conditions effective to produce a stream comprising methanol (MeOH) and dimethyl ether (DME) and optionally unreacted methane; and
an electrolysis unit comprising at least one electrolysis cell and a power source for supplying current to said electrolysis cell, which is fluidly connected to said hydrolysis reactor, and configured to receive an aqueous solution of hydrogen halide (HX_{(aq)}) and to decompose said aqueous solution of hydrogen halide (HX_{(aq)}) into a gaseous hydrogen (H₂) stream and a stream comprising halogen reactant (X₂);
a feed stream supply system for supplying a gaseous feed stream comprising methane to said halogenation reactor;
a halogen supply system for supplying a halogen reactant (X₂) to said halogenation reactor;
an effluent recovery system, configured to recover an effluent stream from said halogenation reactor, and for feeding said recovered effluent stream to said hydrolysis reactor;
a methanol recovery system, configured to recover a stream comprising methanol (MeOH), and dimethyl ether (DME) and optionally unreacted methane from said hydrolysis reactor;
a hydrogen recovery system, configured to recover a gaseous hydrogen (H₂) stream from said electrolysis unit;
a halogen recovery system, configured to recover halogen reactant (X₂) from said electrolysis unit;
a hydrogen halide transfer system, configured to recover an aqueous solution of hydrogen halide (HX_{(aq)}) from said hydrolysis reactor, and to supply said recovered aqueous solution of hydrogen halide (HX_{(aq)}) to the electrolysis unit.

The present process advantageously allows to produce a substantially pure stream of methanol or methanol and DME. The process of the disclosure avoids any direct CO₂ emissions.

Also, in certain embodiments of the present process, a mixture of methanol and dimethyl ether (DME) can be obtained. The present process further allows separating such a mixture into substantially pure streams of methanol and DME. Methanol and DME can both be further used in numerous downstream applications.

In addition, the present process also allows to obtain a substantially pure stream of hydrogen gas as a co-product, which can be further used in numerous downstream applications.

The present disclosure also provides a process for producing methanol, and optionally DME in which no syngas is involved. The production of methanol and DME directly from methane is a key reaction in enabling the switch from petroleum sources to renewable sources for the production of base chemicals. The methane used in the process can come from a renewable source, which makes the production of the methanol and DME and the base chemicals made from said methanol and optional DME compounds, more eco-friendly.

The present process is also highly efficient as it allows to recycle (re-use) reagents and reaction products in the same process, thereby reducing waste and/or pollution.

Moreover, the process allows to produce hydrogen gas by the decomposition of an aqueous solution of hydrogen halide stream by means of electrolysis. Hence, at least part of the energy applied in the present process is based on electric energy, which can be obtained from a renewable source.

The independent and dependent claims set out particular and preferred features of the disclosure. Features from the dependent claims may be combined with features of the independent or other dependent claims as appropriate.

The present disclosure will now be further described. In the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### DETAILED DESCRIPTION OF THE FIGURES

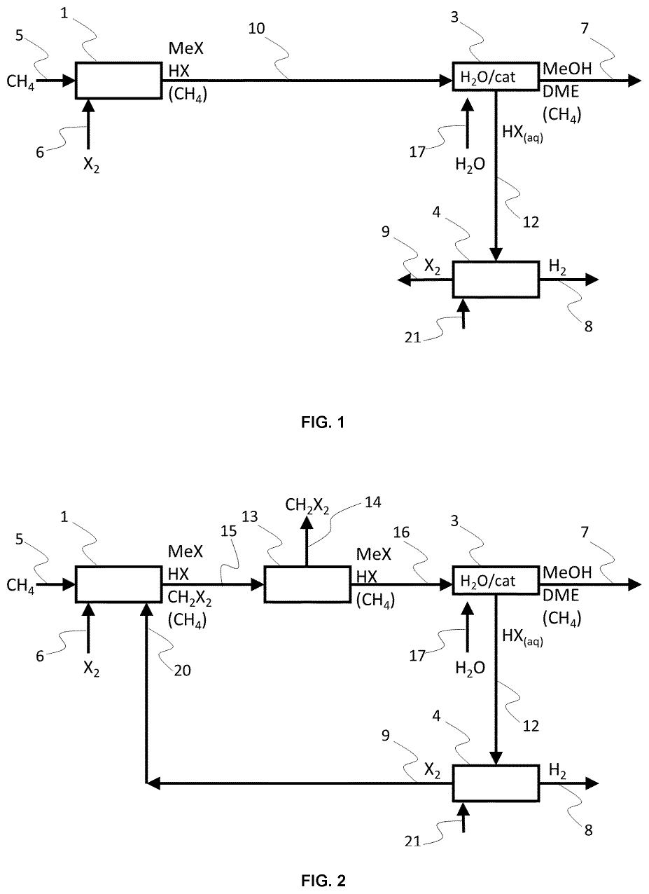

FIG. 1 depicts a process according to an embodiment of the disclosure, and a system according to an embodiment of the disclosure.

FIG. 2 depicts a process according to another embodiment of the disclosure, and a system according to an embodiment of the disclosure.

### DETAILED DESCRIPTION OF THE DISCLOSURE

When describing the disclosure, the terms used are to be construed in accordance with the following definitions, unless a context dictates otherwise.

Unless otherwise defined, all terms used in disclosing the disclosure, including technical and scientific terms, have the meaning as commonly understood by one of ordinary skill in the art to which this disclosure belongs. By means of further guidance, term definitions are included to better appreciate the teaching of the present disclosure.

In the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure, or characteristic described in connection with the embodiment is included in at least one embodiment of the present disclosure. Thus, appearances of the phrases “in one embodiment” or “in an embodiment” in various places throughout this specification are not necessarily all referring to the same embodiment, but may. Furthermore, the particular features, structures, or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure, and form different embodiments, as would be understood by those in the art.

The terms “comprising”, “comprises” and “comprised of” as used herein are synonymous with “including”, “includes” or “containing”, “contains”, and are inclusive or open-ended and do not exclude additional, non-recited members, elements, or process steps. It will be appreciated that the terms “comprising”, “comprises” and “comprised of” as used herein comprise the terms “consisting of”, “consists” and “consists of”.

As used in the specification and the appended claims, the singular forms “a”, “an,” and “the” include plural referents unless the context clearly dictates otherwise. By way of example, “a step” means one step or more than one step.

Unless defined otherwise, all technical and scientific terms used herein have the same meaning as is commonly understood by one of skill in the art.

The recitation of numerical ranges by endpoints includes all integer numbers and, where appropriate, fractions subsumed within that range (e.g., 1 to 5 can include 1, 2, 3, 4, 5 when referring to, for example, a number of elements, and can also include 1.5, 2, 2.75 and 3.80, when referring to, for example, measurements). The recitation of endpoints also includes the end point values themselves (e.g., from 1.0 to 5.0 includes both 1.0 and 5.0). Any numerical range recited herein is intended to include all sub-ranges subsumed therein.

The term “about” as used herein when referring to a measurable value such as a parameter, an amount, a temporal duration, and the like, is meant to encompass variations of +/−10% or less, preferably +/−5% or less, more preferably +/−1% or less, of and from the specified value, insofar such variations are appropriate to perform in the disclosed disclosure. It is to be understood that the value to which the modifier “about” refers is itself also specifically, and preferably, disclosed.

The terms “wt. %”, “vol. %”, or “mol. %” refers to a weight percentage of a component, a volume percentage of a component, or molar percentage of a component, respectively, based on the total weight, the total volume of material, or total moles, that includes the component.

When describing the present disclosure, the terms used are to be construed in accordance with the following definitions, unless a context dictates otherwise.

Whenever the term “substituted” is used in the present disclosure, it is meant to indicate that one or more hydrogens on the atom indicated in the expression using “substituted” is replaced with a selection from the indicated group, provided that the indicated atom's normal valency is not exceeded and that the substitution results in a chemically stable compound, i.e., a compound that is sufficiently robust to survive isolation to a useful degree of purity from a reaction mixture.

The term “alkyl” as a group or part of a group, refers to a hydrocarbyl group of formula CₙH₂ₙ₊₁ wherein n is a number greater than or equal to 1. Alkyl groups may be linear or branched and may be substituted as indicated herein. Generally, alkyl groups of this disclosure comprise from 1 to 20 carbon atoms, preferably from 1 to 18 carbon atoms, preferably from 1 to 12 carbon atoms, preferably from 1 to 10 carbon atoms, preferably from 1 to 6 carbon atoms, more preferably 1, 2, 3, 4, 5, 6 carbon atoms. When a subscript is used herein following a carbon atom, the subscript refers to the number of carbon atoms that the named group may contain. For example, the term “C₁₋₂₀alkyl”, as a group or part of a group, refers to a hydrocarbyl group of formula —CₙH₂ₙ₊₁ wherein n is a number ranging from 1 to 20. Thus, for example, C₁₋₂₀alkyl groups include all linear, or branched alkyl groups having 1 to 20 carbon atoms, and thus includes for example methyl, ethyl, n-propyl, i-propyl, 2-methyl-ethyl, butyl and its isomers (e.g., n-butyl, i-butyl and t-butyl); pentyl and its isomers, hexyl and its isomers, heptyl and its isomers, octyl and its isomers, nonyl and its isomers, decyl and its isomers, undecyl and its isomers, dodecyl and its isomers, tridecyl and its isomers, tetradecyl and its isomers, pentadecyl and its isomers, hexadecyl and its isomers, heptadecyl and its isomers, octadecyl and its isomers, and the like. For example, C₁₋₁₀alkyl includes all linear, or branched alkyl groups having 1 to 10 carbon atoms, and thus includes for example methyl, ethyl, n-propyl, i-propyl, 2-methyl-ethyl, butyl and its isomers (e.g., n-butyl, i-butyl, and t-butyl); pentyl and its isomers, hexyl and its isomers, heptyl and its isomers, octyl and its isomers, nonyl and its isomers, decyl and its isomers and the like. For example, C₁₋₆alkyl includes all linear, or branched alkyl groups having 1 to 6 carbon atoms, and thus includes for example methyl, ethyl, n-propyl, i-propyl, 2-methyl-ethyl, butyl and its isomers (e.g., n-butyl, i-butyl, and t-butyl); pentyl and its isomers, hexyl and its isomers. In some embodiments, non-limiting examples of alkyl groups include for instance methyl, ethyl, propyl, iso-propyl, n-butyl, isobutyl, sec-butyl, t-butyl, pentyl, isopentyl, 2,2-dimethyl-propyl, hexyl, 2,3-dimethyl-2-butyl, heptyl, 2,2-dimethyl-3-pentyl, 2-methyl-2-hexyl, octyl, 4-methyl-3-heptyl, nonyl, decyl, undecyl and dodecyl groups. As used herein and unless otherwise stated, the term “halo” or “halogen”, as a group or part of a group, is generic for any atom selected from the group consisting of fluorine (F), chlorine (Cl), bromine (Br), iodine (I) and astatine (At).

Preferred statements (features) and embodiments and uses of this disclosure are set herein below. Each statement and embodiment of the disclosure so defined may be combined with any other statement and/or embodiment unless clearly indicated to the contrary. In particular, any feature indicated as being preferred or advantageous may be combined with any other feature or features or statements indicated as being preferred or advantageous. Hereto, the present disclosure is in particular captured by any one or any combination of one or more of the below-numbered statements and embodiments, with any other aspect and/or embodiment.

1. A process for producing methanol (MeOH) and hydrogen (H₂) from methane, comprising the steps of:
a) providing a gaseous feed stream comprising methane;
b) reacting said gaseous feed stream with at least one halogen reactant (X₂), under reaction conditions effective to produce an effluent stream comprising methyl halide (MeX), hydrogen halide (HX), optionally polyhalogenated alkanes, and optionally unreacted methane;
c) recovering an effluent stream comprising methyl halide (MeX), hydrogen halide (HX), optionally polyhalogenated alkanes, and optionally unreacted methane;
d) reacting the effluent stream obtained in step c) with water and at least one catalyst, under reaction conditions effective to produce:
   an aqueous solution of hydrogen halide (HX_{(aq)}), and
   a methanol stream comprising methanol (MeOH), and optionally dimethyl ether (DME) and/or optionally unreacted methane,
e) decomposing by means of electrolysis said aqueous solution of hydrogen halide (HX_{(aq)}) under conditions effective to produce a gaseous hydrogen (H₂) stream and a stream comprising halogen reactant (X₂); and,
f) optionally, recovering methanol from said methanol (MeOH) stream.

More particularly, the present disclosure thereto provides a process for producing methanol (MeOH), dimethyl ether (DME), and hydrogen (H₂) from methane, comprising the steps of:
a) providing a gaseous feed stream comprising methane;
b) reacting said gaseous feed stream with at least one halogen reactant (X₂), under reaction conditions effective to produce an effluent stream comprising methyl halide (MeX), hydrogen halide (HX), optionally polyhalogenated alkanes, and optionally unreacted methane;
c) recovering an effluent stream comprising methyl halide (MeX), hydrogen halide (HX), optionally polyhalogenated alkanes, and optionally unreacted methane;
d) reacting the effluent stream obtained in step c) with water and an organic base, under reaction conditions effective to produce:
   an aqueous solution of hydrogen halide (HX_{(aq)}), and
   a methanol stream comprising methanol (MeOH) and dimethyl ether (DME) and optionally unreacted methane, and,
e) decomposing by means of electrolysis said aqueous solution of hydrogen halide (HX(aq)) under conditions effective to produce a gaseous hydrogen (H₂) stream and a stream comprising halogen reactant (X₂); and,
f) optionally, recovering methanol from said methanol (MeOH) stream and/or recovering dimethyl ether (DME) from said methanol (MeOH) stream.

2. The process according to statement 1, wherein the process comprises the step of separating said polyhalogenated alkanes formed in step b) from said effluent stream, preferably prior to reacting the effluent stream with water and at least one catalyst.

3. The process according to any one of statements 1 to 2, wherein said polyhalogenated alkanes comprise polyhalogenated methanes and optionally polyhalogenated C₂₊ alkanes.

4. A process for producing methanol (MeOH) and hydrogen (H₂) from methane, preferably according to any one of statements 1 to 3, comprising the steps of:
a) providing a gaseous feed stream comprising methane;
b) reacting said gaseous feed stream with at least one halogen reactant (X₂), under reaction conditions effective to produce an effluent stream comprising methyl halide (MeX), hydrogen halide (HX), optionally polyhalogenated alkanes, and optionally unreacted methane;
c) separating from the effluent stream obtained in step b):
   c1) a mono-halide stream, comprising methyl halide (MeX) and hydrogen halide (HX), and optionally comprising unreacted methane; and,
   c2) a polyhalogenated alkanes stream,
d) reacting the mono-halide stream separated in step c1), with water and at least one catalyst, under reaction conditions effective to produce:
   d1) an aqueous solution of hydrogen halide (HX_{(aq)}), and
   d2) a methanol stream comprising methanol (MeOH), and optionally dimethyl ether (DME), and/or optionally unreacted methane,
e) decomposing by means of electrolysis said aqueous solution of hydrogen halide (HX_{(aq)}) produced in step d1) under conditions effective to produce a gaseous hydrogen (H₂) stream and a stream comprising halogen reactant (X₂); and,
f) optionally, recovering methanol from said methanol (MeOH) stream.

For example, the process comprises the steps of:
a) providing a gaseous feed stream comprising methane;
b) reacting said gaseous feed stream with at least one halogen reactant (X₂), under reaction conditions effective to produce an effluent stream comprising methyl halide (MeX), hydrogen halide (HX), optionally polyhalogenated alkanes, and optionally unreacted methane;
c) separating from the effluent stream obtained in step b):
   c1) a mono-halide stream, comprising methyl halide (MeX) and hydrogen halide (HX), and optionally comprising unreacted methane; and,
   c2) a polyhalogenated alkanes stream,
d) reacting the mono-halide stream separated in step c1), with water and an organic base, under reaction conditions effective to produce:
   d1) an aqueous solution of hydrogen halide (HX_{(aq)}), and
   d2) a methanol stream comprising methanol (MeOH) and dimethyl ether (DME), and optionally unreacted methane,
e) decomposing by means of electrolysis said aqueous solution of hydrogen halide (HX_{(aq)}) produced in step d1) under conditions effective to produce a gaseous hydrogen (H₂) stream and a stream comprising halogen reactant (X₂); and, optionally, recovering methanol from said methanol (MeOH) stream and/or recovering dimethyl ether (DME) from said methanol (MeOH) stream.

5. The process according to any one of the previous statements, wherein the step of separating said polyhalogenated alkanes formed in step b) from said effluent stream is performed prior to separating said mono-halide stream from said effluent stream.

6. The process according to any one of the previous statements, wherein said halogen reactant is selected form the group consisting of bromine (Br₂), chlorine (Cl₂), fluorine (F₂), iodine (I₂), and astatine (At₂).

7. The process according to any one of the previous statements, wherein said halogen reactant is bromine (Br₂).

8. The process according to any one of the previous statements, wherein step b) and d) are performed in separate reaction steps, preferably in separate reactors or separate reaction zones.

9. The process according to any one of the previous statements, wherein the temperature during step b) is at least 150° C., preferably at least 200° C., preferably at least 250° C., preferably at least 300° C., preferably at least 350° C., preferably at least 380° C.

10. The process according to any one of the previous statements, wherein the temperature during step b) is at most 900° C., preferably at most 800° C., preferably at most 700° C., preferably at most 600° C., preferably at most 550° C., preferably at most 520° C.

11. The process according to any one of the previous statements, wherein the temperature during step b) is at least 150° C. to at most 900° C., preferably at least 200° C. to at most 800° C., preferably at least 250° C. to at most 700° C., preferably at least 300° C. to at most 600° C., preferably at least 350° C. to at most 550° C., preferably at least 380° C. to at most 520° C.

12. The process according to any one of the previous statements, wherein the pressure during step b) is at least 1.0 bar, preferably at least 1.5 bar, preferably at least 2.0 bar, preferably at least 2.5 bar, preferably at least 3.0 bar, preferably at least 5.0 bar, preferably at least 10.0 bar.

13. The process according to any one of the previous statements, wherein the pressure during step b) is at most 50.0 bar, preferably at most 45.0 bar, preferably at most 40.0 bar, preferably at most 35.0 bar, preferably at most 30.0 bar, preferably at most 25.0 bar, preferably at most 20.0 bar.

14. The process according to any one of the previous statements, wherein the pressure during step b) is at least 1.0 bar to at most 50.0 bar, preferably at least 1.5 bar to at most 45.0 bar, preferably at least 2.0 bar to at most 40.0 bar, preferably at least 2.5 bar to at most 35.0 bar, preferably at least 3.0 bar to at most 30.0 bar, preferably at least 5.0 bar to at most 25.0 bar, preferably at least 10.0 bar to at most 20.0 bar.

15. The process according to any one of the previous statements, wherein in step b) the gaseous feed stream is contacted with the halogen reactant (X₂) for at least 0.1 min to at most 2.0 min, preferably for at least 0.2 min to at most 1.7 min, preferably for at least 0.5 min to at most 1.5 min, preferably for at least 0.7 min to at most 1.2 min.

16. The process according to any one of the previous statements, wherein polyhalogenated alkanes are formed during step b), preferably wherein the formed polyhalogenated alkanes formed during step b) comprise polyhalogenated methane and optionally polyhalogenated C₂₊ alkanes.

17. The process according to any one of the previous statements, wherein polyhalogenated alkanes formed in step b) of the process comprise methylene dihalide (CH₂X₂).

18. The process according to any one of the previous statements, wherein polyhalogenated alkanes formed in step b) of the process are separated from the effluent stream by distillation.

19. The process according to any one of the previous statements, wherein polyhalogenated alkanes formed in step b) of the process are separated from the effluent stream as a polyhalogenated methane stream and a polyhalogenated C₂₊ alkanes stream, preferably by distillation.

20. The process according to any one of the previous statements, wherein C₂₊ alkyl monohalides are formed in step b).

21. The process according to any one of the previous statements, wherein the process comprises the step of separating C₂₊ alkyl monohalides formed in step b) of the process from said effluent stream.

22. The process according to any one of the previous statements, wherein the process comprises separating said C₂₊ alkyl monohalides formed in step b) from said effluent stream prior to separating the mono-halide stream comprising methyl halide (MeX) and hydrogen halide (HX).

23. The process according to any one of the previous statements, wherein the process comprises separating said C₂₊ alkyl monohalides formed in step b) from said effluent stream after separating said polyhalogenated alkanes from said effluent stream.

24. A process for producing methanol (MeOH) and hydrogen from methane, preferably according to any one of the previous statements, comprising the steps of:
a) providing a gaseous feed stream comprising methane;
b) reacting said gaseous feed stream with at least one halogen reactant (X₂), under reaction conditions effective to produce an effluent stream comprising methyl halide (MeX), hydrogen halide (HX), polyhalogenated alkanes, C₂₊ alkyl monohalides, and optionally unreacted methane;
c) separating from the effluent stream obtained in step b) the following substreams:
   c1) a mono-halide stream, comprising methyl halide (MeX), hydrogen halide (HX), and optionally comprising unreacted methane; and,
   c2) a polyhalogenated alkanes stream; and
   c2) a C₂₊ alkyl monohalides stream;
d) reacting the mono-halide stream separated in step c1), with water and at least one catalyst, under reaction conditions effective to produce:
   d1) an aqueous solution of hydrogen halide (HX_{(aq)}), and
   d2) a methanol stream comprising methanol (MeOH), and optionally dimethyl ether (DME), and/or optionally unreacted methane,
e) decomposing by means of electrolysis said aqueous solution of hydrogen halide (HX_{(aq)}) produced in step d1) under conditions effective to produce a gaseous hydrogen (H₂) stream and a stream comprising halogen reactant (X₂); and,
f) optionally, recovering methanol from said methanol (MeOH) stream.

25. The process according to any one of the previous statements, wherein methanol stream comprising methanol (MeOH), and optionally dimethyl ether (DME), and/or optionally unreacted methane is separated from the aqueous solution of hydrogen halide by means of distillation.

26. The process according to any one of the previous statements, wherein the stream comprising methanol and/or dimethyl ether recovered after step d) comprises, based on the total amount of the stream, at least 75.0 mol %, such as least 80.0 mol %, or at least 85.0 mol %, or at least 95.0 mol % of methanol and dimethyl ether in combination.

27. The process according to any one of the previous statements, wherein C₂₊ alkyl monohalides formed in step b) of the process are separated from the effluent stream by distillation.

28. The process according to any one of the previous statements, wherein mono-halide stream separated in step c) is a gaseous stream.

29. The process according to any one of the previous statements, wherein the mono-halide stream recovered in step c) is a gaseous stream.

30. The process according to any one of the previous statements, wherein the mono-halide stream separated in step c) is essentially free of polyhalogenated alkanes.

31. The process according to any one of the previous statements, wherein the mono-halide stream separated in step c) is essentially free of C₂₊ alkyl monohalides.

32. The process according to any one of the previous statements, wherein step c) comprises separating from the effluent stream:
c1) a mono-halide stream, comprising methyl halide (MeX) and hydrogen halide (HX), and optionally comprising unreacted methane; and,
c2) a polyhalogenated alkanes stream, comprising polyhalogenated methane stream and optionally polyhalogenated C₂₊ alkanes stream; and,
c3) optionally a C₂₊ alkyl monohalides stream.

33. The process according to any one of the previous statements, wherein the mono-halide stream comprises unreacted methane.

34. The process according to any one of the previous statements, wherein the mono-halide stream comprises C₂₊ alkyl monohalides.

35. The process according to any one of the previous statements, wherein the gaseous feed stream comprises at least 75.0 vol % methane, preferably at least 80.0 vol % methane, preferably at least 85.0 vol % methane, preferably at least 90.0 vol % methane, preferably at least 95.0 vol % methane, preferably at least 98.0 vol % methane, preferably at least 99.0 vol % methane, preferably at least 99.9 vol % methane, based on the total volume of the gaseous feed stream.

36. The process according to any one of the previous statements, wherein the gaseous feed stream comprises natural gas, a biogas, a refinery gas, or any mixture thereof.

37. The process according to any one of the previous statements, wherein the gaseous feed stream applied in step a) of the process is substantially free of sulphur species, e.g. it contains less than 1.0 mol %, or less than 0.5 mol %, or less than 0.1 mol %, or less than 0.01 mol %, or less than 0.001 mol %, or less than 0.0001 mol %, or less than 0.00001 mol % of sulphur species.

38. The process according to any one of the previous statements, wherein in step d) the effluent stream or the recovered mono-halide stream is reacted with water, wherein the water comprises at least one catalyst.

39. The process according to any one of the previous statements, wherein at least one catalyst is a base, preferably an organic base.

40. The process according to any one of the previous statements, wherein at least one catalyst is an N-containing polymer, and preferably a polymer comprising at least one amino group, pyridine ring, pyridazine ring, pyrimidine ring, pyrazine ring, triazine ring, pyrazole ring, imidazole ring, oxazole ring, isoxazole ring, thiazole ring, isothiazole ring, triazole ring, oxadiazole ring, or any combinations thereof.

41. The process according to any one of the previous statements, wherein in step d) the mono-halide stream is contacted with water and the catalyst, for at least 0.1 min to at most 600.0 min, preferably for at least 0.2 min to at most 400.0 min, preferably for at least 0.5 min to at most 200.0 min, preferably for at least 1.0 min to at most 100.0 min, preferably for at least 2.0 min to at most 75.0 min, preferably for at least 3.0 min to at most 60.0 min, preferably for at least 4.0 min to at most 30.0 min, preferably for at least 5.0 min to at most 15.0 min.

42. The process according to any one of the previous statements, wherein the temperature during step d) is at least 50° C., preferably at least 70° C., preferably at least 80° C., preferably at least 90° C., preferably at least 100° C., preferably at least 120° C.

43. The process according to any one of the previous statements, wherein the temperature during step d) is at most 500° C., preferably at most 450° C., preferably at most 400° C., preferably at most 350° C., preferably at most 300° C., preferably at most 250° C.

44. The process according to any one of the previous statements, wherein the temperature during step d) is at least 50° C. to at most 500° C., preferably at least 70° C. to at most 450° C., preferably at least 80° C. to at most 400° C., preferably at least 90° C. to at most 350° C., preferably at least 100° C. to at most 300° C., preferably at least 120° C. to at most 250° C.

45. The process according to any one of the previous statements, wherein the pressure during step d) is at least 1.0 bar, preferably at least 1.2 bar, preferably at least 1.5 bar, preferably at least 1.7 bar, preferably at least 2.0 bar, preferably at least 5.0 bar, preferably at least 10.0 bar.

46. The process according to any one of the previous statements, wherein the pressure during step d) is at most 40.0 bar, preferably at most 35.0 bar, preferably at most 30.0 bar, preferably at most 25.0 bar, preferably at most 20.0 bar, preferably at most 15.0 bar, preferably at most 10.0 bar.

47. The process according to any one of the previous statements, wherein the pressure during step d) is at least 1.0 bar to at most 40.0 bar, preferably at least 1.2 bar to at most 35.0 bar, preferably at least 1.5 bar to at most 30.0 bar, preferably at least 1.7 bar to at most 25.0 bar, preferably at least 2.0 bar to at most 20.0 bar preferably at least 5.0 bar to at most 20.0 bar, preferably at least 10.0 bar to at most 15.0 bar.

48. The process according to any one of the previous statements, wherein polyhalogenated alkanes are converted to their corresponding alcohols, preferably by hydrogenation said polyhalogenated alkanes into monohalides followed by contacting said monohalides with a solid metal hydroxide (MOH₍ₛ₎), under reaction conditions effective to produce the corresponding alcohols.

49. The process according to any one of the previous statements, wherein C₂₊ alkyl monohalides stream is converted to their corresponding alcohols, preferably by contacting said C₂₊ alkyl monohalides with a solid metal hydroxide (MOH₍ₛ₎), under reaction conditions effective to produce the corresponding alcohols.

50. The process according to any one of the previous statements, wherein the process further comprises the step:
e) recovering at least a part of said at least one catalyst after step d) or during step e) of the process, and supplying said recovered catalyst in step d) of the process.

51. The process according to any one of the previous statements, further comprising the step of returning the halogen reactant (X₂) obtained in step e), to step b) of the process.

52. The process according to any one of the previous statements, wherein halogen reactant (X₂) obtained in step e) is purified before being fed into step b), and preferably purified by condensation of the halogen reactant (X₂).

53. The process according to any one of the previous statements, wherein the halogen reactant (X₂) obtained in step e), is dried before being fed into step b), preferably dried over molecular sieves.

54. The process according to any one of the previous statements, wherein step e) of said process comprises supplying an aqueous solution of hydrogen halide (HX) to an electrolysis cell containing positive and negative electrodes, and decomposing said hydrogen halide electrolytically by maintaining an electrical potential from about 0.5 to 2.5 V between said electrodes.

55. The process according to any one of the previous statements, wherein step e) of said process comprises supplying an aqueous solution of hydrogen halide (HX) to an electrolysis cell containing positive and negative electrodes, and decomposing said hydrogen halide electrolytically by maintaining a current density from about 100 to 800 mA/cm² between said electrodes.

56. The process according to any one of the previous statements, wherein said electrolysis cell is a polymer electrolyte membrane cell (PEM) containing at least a proton-conductive membrane.

57. The process according to any one of the previous statements, wherein said hydrogen halide is decomposed in step e) at a temperature of from 20 to 95° C.

58. The process according to any one of the previous statements, wherein said hydrogen halide is decomposed in step e) at a pressure of from 1 to 50 bar.

59. The process according to any one of the previous statements, wherein a solution comprising catalyst is recovered in step e), preferably said recovered solution comprising catalyst is returned to step d).

60. The process according to any one of the previous statements, wherein the gaseous hydrogen (H₂) stream generated in step e) comprises at least 90.0 mol %, such as at least 95.0 mol %, or at least 99.0 mol %, or at least 99.5 mol %, or at least 99.9 mol % hydrogen.

61. A process for producing methanol (MeOH) and hydrogen from methane, according to any one of the previous statements comprising the steps of:
a) providing a gaseous feed stream comprising methane,
b) reacting said gaseous feed stream with bromide (Br₂), under reaction conditions effective to produce an effluent stream comprising hydrogen bromide (HBr), methyl bromide (MeBr), optionally polybrominated alkanes, such as polybrominated methanes selected from CH₂Br₂, CHBr₃ and/or CBr₄; and optionally unreacted methane,
c) recovering an effluent stream comprising methyl bromide (MeBr), hydrogen bromide (HBr), optionally polybrominated alkanes, such as polybrominated methanes selected from CH₂Br₂, CHBr₃ and/or CBr₄; and optionally unreacted methane optionally polybrominated alkanes, and optionally unreacted methane;
d) reacting the effluent stream obtained in step c) with water and at least one catalyst, under reaction conditions effective to produce:
   an aqueous solution of hydrogen bromide (HBr_{(aq)}), and
   a methanol stream comprising methanol (MeOH), and optionally dimethyl ether (DME) and/or optionally unreacted methane,
e) decomposing by means of electrolysis said aqueous solution of hydrogen bromide under conditions effective to produce a gaseous hydrogen (H₂) stream and a stream comprising bromine (Br₂); and,
f) optionally, recovering methanol from said methanol (MeOH) stream, and
g) optionally, recovering at least part of the catalyst after step d) or during step e), and providing the recovered catalyst in step d).

62. A process for producing methanol (MeOH) and hydrogen from methane, according to any one of the previous statements comprising the steps of:
a) providing a gaseous feed stream comprising methane,
b) reacting said gaseous feed stream with bromide (Br₂), under reaction conditions effective to produce an effluent stream comprising hydrogen bromide (HBr), methyl bromide (MeBr), optionally polybrominated alkanes, such as polybrominated methane selected from CH₂Br₂, CHBr₃ and/or CBr₄; and optionally unreacted methane,
c) separating from the effluent stream obtained in step b):
   (i) a mono-bromide stream, comprising methyl bromide (MeBr) and hydrogen bromide (HBr), and optionally comprising unreacted methane; and,
   (ii) a polybrominated alkanes stream, comprising polybrominated methane;
d) reacting the mono-bromide steam separated in step c1), with water and at least one catalyst, under reaction conditions effective to produce:
   d1) an aqueous solution of hydrogen bromide (HBr_{(aq)}), and
   d2) a methanol stream comprising methanol (MeOH), and optionally dimethyl ether (DME), and/or optionally unreacted methane,
e) decomposing by means of electrolysis said aqueous solution of hydrogen bromide (HBr_{(aq)}) obtained in step d1) under conditions effective to produce a gaseous hydrogen (H₂) stream and a stream comprising bromine (Br₂);
f) optionally recovering methanol from said methanol (MeOH) stream; and,
g) optionally, recovering at least partially the catalyst after step d) or during step e), and providing the recovered catalyst in step d).

63. The process according to any one of the previous statements, wherein methanol stream comprising methanol (MeOH), and optionally dimethyl ether (DME), and/or optionally unreacted methane is separated from the aqueous solution of hydrogen bromide by means of distillation.

64. The process according to any one of the previous statements, wherein the process further comprises a step of removing, preferably distilling, unreacted methane from the effluent stream.

65. The process according to any one of the previous statements, wherein the process further comprises a step of removing, preferably distilling, unreacted methane from the mono-halide stream.

66. The process according to any one of the previous statements, wherein the process further comprises a step of removing, preferably distilling, unreacted methane, from the methanol stream produced in step d2).

67. The process according to any one of the previous statements, wherein removed unreacted methane is recycled or fed into step a) and/or step b).

68. The process according to any one of the previous statements, wherein the process is a continuous process.

69. System for producing methanol (MeOH) and hydrogen (H₂) from methane, comprising;
a halogenation reactor, configured to react a gaseous feed stream comprising methane with at least one halogen reactant (X₂) into an effluent stream comprising methyl halide (MeX), and hydrogen halide (HX), and optionally unreacted methane,
optionally, a polyhalogenated alkane removal unit, configured to receive an effluent stream from said halogenation reactor and configured to separate from said effluent stream:
   (i) a polyhalogenated alkane stream, comprising polyhalogenated alkanes; and,
   (ii) a mono-halide stream, comprising methyl halide (MeX) and hydrogen halide (HX), and optionally comprising unreacted methane; and,
a hydrolysis reactor, which is fluidly connected:
   to said halogenation reactor and configured to receive an effluent stream from said halogenation reactor; or,
   to said polyhalogenated alkane removal unit and configured to receive a mono-halide stream from said polyhalogenated alkane removal unit;
   and, wherein said hydrolysis reactor is configured to react the effluent stream or the mono-halide stream, with water and at least one catalyst, under reaction conditions effective to produce (i) an aqueous solution of hydrogen halide (HX_{(aq)}), and (ii) a stream comprising methanol (MeOH) and optionally dimethyl ether (DME) and/or optionally unreacted methane; and
an electrolysis unit comprising at least one electrolysis cell and a power source for supplying current to said electrolysis cell, which is fluidly connected to said hydrolysis reactor, and configured to receive an aqueous solution of hydrogen halide (HX_{(aq)}) and to decompose said aqueous solution of hydrogen halide (HX_{(aq)}) into a gaseous hydrogen (H₂) stream and a stream comprising halogen reactant (X₂);
a feed stream supply system for supplying a gaseous feed stream comprising methane to said halogenation reactor;
a halogen supply system for supplying a halogen reactant (X₂) to said halogenation reactor;
an effluent recovery system, configured to recover an effluent stream from said halogenation reactor, and for feeding said recovered effluent stream to said optional polyhalogenated alkane removal unit, or to said hydrolysis reactor;
a methanol recovery system, configured to recover a stream comprising methanol (MeOH), and optionally dimethyl ether (DME), and/or optionally unreacted methane from said hydrolysis reactor;
a hydrogen recovery system, configured to recover a gaseous hydrogen (H₂) stream from said electrolysis unit;
a halogen recovery system, configured to recover halogen reactant (X₂) from said electrolysis unit;
a hydrogen halide transfer system, configured to recover an aqueous solution of hydrogen halide (HX_{(aq)}) from said hydrolysis reactor, and to supply said recovered aqueous solution of hydrogen halide (HX_{(aq)}) to the electrolysis unit;
optionally, a mono-halide transfer system, configured to recover a mono-halide stream from said polyhalogenated alkane removal unit, and to supply said mono-halide stream to the hydrolysis reactor,
optionally, a water supply line, for supplying water, preferably water and at least one catalyst, to said hydrolysis reactor;
optionally, a C₂₊ alkyl monohalide removal unit, configured to receive effluent stream from said halogenation reactor and to remove C₂₊ alkyl monohalides from said effluent stream.

In particular, the disclosure also provides a system for producing methanol (MeOH), dimethyl ether (DME) and hydrogen (H₂) from methane, comprising;
a halogenation reactor, configured to react a gaseous feed stream comprising methane with at least one halogen reactant (X₂) into an effluent stream comprising methyl halide (MeX), and hydrogen halide (HX), and optionally unreacted methane; and,
a hydrolysis reactor, which is fluidly connected to said halogenation reactor and configured to receive an effluent stream from said halogenation reactor; and, wherein said hydrolysis reactor is configured to react the effluent stream with water and an organic base, under reaction conditions effective to produce a stream comprising methanol (MeOH) and dimethyl ether (DME) and optionally unreacted methane; and
an electrolysis unit comprising at least one electrolysis cell and a power source for supplying current to said electrolysis cell, which is fluidly connected to said hydrolysis reactor, and configured to receive an aqueous solution of hydrogen halide (HX_{(aq)}) and to decompose said aqueous solution of hydrogen halide (HX_{(aq)}) into a gaseous hydrogen (H₂) stream and a stream comprising halogen reactant (X₂);
a feed stream supply system for supplying a gaseous feed stream comprising methane to said halogenation reactor;
a halogen supply system for supplying a halogen reactant (X₂) to said halogenation reactor;
an effluent recovery system, configured to recover an effluent stream from said halogenation reactor, and for feeding said recovered effluent stream to said hydrolysis reactor;
a methanol recovery system, configured to recover a stream comprising methanol (MeOH), and dimethyl ether (DME) and optionally unreacted methane from said hydrolysis reactor;
a hydrogen recovery system, configured to recover a gaseous hydrogen (H₂) stream from said electrolysis unit;
a halogen recovery system, configured to recover halogen reactant (X₂) from said electrolysis unit;
a hydrogen halide transfer system, configured to recover an aqueous solution of hydrogen halide (HX_{(aq)}) from said hydrolysis reactor, and to supply said recovered aqueous solution of hydrogen halide (HX_{(aq)}) to the electrolysis unit.

In a first aspect, the disclosure provides a process for producing methanol (MeOH) and hydrogen (H₂) from methane.

A process of the disclosure comprises the following steps of:
a) providing a gaseous feed stream comprising methane;
b) reacting said gaseous feed stream with at least one halogen reactant (X₂), under reaction conditions effective to produce an effluent stream comprising methyl halide (MeX), hydrogen halide (HX), optionally polyhalogenated alkanes, and optionally unreacted methane;
c) recovering an effluent stream comprising methyl halide (MeX), hydrogen halide (HX), optionally polyhalogenated alkanes, and optionally unreacted methane;
d) reacting the effluent stream obtained in step c) with water and at least one catalyst, under reaction conditions effective to produce:
   an aqueous solution of hydrogen halide (HX_{(aq)}), and
   a methanol stream comprising methanol (MeOH), and optionally dimethyl ether (DME) and/or optionally unreacted methane,
e) decomposing by means of electrolysis said aqueous solution of hydrogen halide (HX_{(aq)}) under conditions effective to produce a gaseous hydrogen (H₂) stream and a stream comprising halogen reactant (X₂); and,
f) optionally, recovering methanol from said methanol (MeOH) stream.

For example, a process of the disclosure comprises the following steps of:
a) providing a gaseous feed stream comprising methane;
b) reacting said gaseous feed stream with at least one halogen reactant (X₂), under reaction conditions effective to produce an effluent stream comprising methyl halide (MeX), hydrogen halide (HX), optionally polyhalogenated alkanes, and optionally unreacted methane;
c) recovering an effluent stream comprising methyl halide (MeX), hydrogen halide (HX), optionally polyhalogenated alkanes, and optionally unreacted methane;
d) reacting the effluent stream obtained in step c) with water and an organic base, under reaction conditions effective to produce:
   an aqueous solution of hydrogen halide (HX_{(aq)}), and
   a methanol stream comprising methanol (MeOH), and dimethyl ether (DME) and optionally unreacted methane,
e) decomposing by means of electrolysis said aqueous solution of hydrogen halide (HX_{(aq)}) under conditions effective to produce a gaseous hydrogen (H₂) stream and a stream comprising halogen reactant (X₂); and,
f) optionally, recovering methanol from said methanol (MeOH) stream and/or recovering dimethyl ether (DME) from said methanol (MeOH) stream.

In certain embodiments, a process of the disclosure for producing methanol (MeOH) and hydrogen (H₂) comprises the steps of
a) providing a gaseous feed stream comprising methane;
b) reacting said gaseous feed stream with at least one halogen reactant (X₂), under reaction conditions effective to produce an effluent stream comprising methyl halide (MeX), hydrogen halide (HX), optionally polyhalogenated alkanes, and optionally unreacted methane;
c) separating from the effluent stream obtained in step b):
   c1) a mono-halide stream, comprising methyl halide (MeX) and hydrogen halide (HX), and optionally comprising unreacted methane; and,
   c2) a polyhalogenated alkanes stream,
d) reacting the mono-halide stream separated in step c1), with water and at least one catalyst, under reaction conditions effective to produce:
   d1) an aqueous solution of hydrogen halide (HX_{(aq)}), and
   d2) a methanol stream comprising methanol (MeOH), and optionally dimethyl ether (DME), and/or optionally unreacted methane,
e) decomposing by means of electrolysis said aqueous solution of hydrogen halide (HX_{(aq)}) produced in step d1) under conditions effective to produce a gaseous hydrogen (H₂) stream and a stream comprising halogen reactant (X₂); and,
f) optionally, recovering methanol from said methanol (MeOH) stream.

For example, a process of the disclosure for producing methanol (MeOH), dimethyl ether (DME) and hydrogen (H₂) comprises the steps of
a) providing a gaseous feed stream comprising methane;
b) reacting said gaseous feed stream with at least one halogen reactant (X₂), under reaction conditions effective to produce an effluent stream comprising methyl halide (MeX), hydrogen halide (HX), optionally polyhalogenated alkanes, and optionally unreacted methane;
c) separating from the effluent stream obtained in step b):
   c1) a mono-halide stream, comprising methyl halide (MeX) and hydrogen halide (HX), and optionally comprising unreacted methane; and,
   c2) a polyhalogenated alkanes stream,
d) reacting the mono-halide stream separated in step c1), with water and an organic base, under reaction conditions effective to produce:
   d1) an aqueous solution of hydrogen halide (HX_{(aq)}), and
   d2) a methanol stream comprising methanol (MeOH), and dimethyl ether (DME), and optionally unreacted methane,
e) decomposing by means of electrolysis said aqueous solution of hydrogen halide (HX_{(aq)}) produced in step d1) under conditions effective to produce a gaseous hydrogen (H₂) stream and a stream comprising halogen reactant (X₂); and,
f) optionally, recovering methanol from said methanol (MeOH) stream and/or recovering dimethyl ether (DME) from said methanol (MeOH) stream.

In certain embodiments of a process of the disclosure, the process comprises the further step of separating said polyhalogenated alkanes formed in step b) from said effluent stream, preferably prior to reacting the effluent stream with water and at least one catalyst, which is preferably an organic base.

Said polyhalogenated alkanes preferably comprise polyhalogenated methanes (as defined herein) and optionally polyhalogenated C₂₊ alkanes (as defined herein).

In certain embodiments, a process of the disclosure comprises the steps of
a) providing a gaseous feed stream comprising methane;
b) reacting said gaseous feed stream with at least one halogen reactant (X₂), under reaction conditions effective to produce an effluent stream comprising thereby producing an effluent stream comprising hydrogen halide (HX), methyl halide (MeX), optionally, polyhalogenated alkanes, such as polyhalogenated methanes, as defined herein, and optionally unreacted methane,
c) optionally, separating from the effluent stream obtained in step b):
   c1) a mono-halide stream, comprising methyl halide (MeX) and hydrogen halide (HX), optionally comprising unreacted methane; and,
   c2) a polyhalogenated alkanes stream, comprising polyhalogenated alkanes, such as polyhalogenated methanes, as defined herein,
d) reacting the effluent stream obtained in step b), or the mono-halide stream separated in step c), with water and at least one catalyst, under reaction conditions effective to produce:
   d1) a stream of an aqueous solution of hydrogen halide (HX_{(aq)}), and
   d2) a methanol stream comprising methanol (MeOH), and optionally dimethyl ether (DME), and/or optionally unreacted methane,
e) decomposing by means of electrolysis said aqueous solution of hydrogen halide (HX_{(aq)}) produced in step d1) under conditions effective to produce a gaseous hydrogen (H₂) stream and a stream comprising halogen reactant (X₂); and,
f) optionally, recovering methanol from said methanol (MeOH) stream.

For example, a process of the disclosure comprises the steps of
a) providing a gaseous feed stream comprising methane;
b) reacting said gaseous feed stream with at least one halogen reactant (X₂), under reaction conditions effective to produce an effluent stream comprising thereby producing an effluent stream comprising hydrogen halide (HX), methyl halide (MeX), optionally, polyhalogenated alkanes, such as polyhalogenated methanes, as defined herein, and optionally unreacted methane,
c) optionally, separating from the effluent stream obtained in step b):
   c1) a mono-halide stream, comprising methyl halide (MeX) and hydrogen halide (HX), optionally comprising unreacted methane; and,
   c2) a polyhalogenated alkanes stream, comprising polyhalogenated alkanes, such as polyhalogenated methanes, as defined herein,
d) reacting the effluent stream obtained in step b), or the mono-halide stream separated in step c), with water and an organic base, under reaction conditions effective to produce:
   d1) a stream of an aqueous solution of hydrogen halide (HX_{(aq)}), and
   d2) a methanol stream comprising methanol (MeOH), and dimethyl ether (DME), and optionally unreacted methane,
e) decomposing by means of electrolysis said aqueous solution of hydrogen halide (HX_{(aq)}) produced in step d1) under conditions effective to produce a gaseous hydrogen (H₂) stream and a stream comprising halogen reactant (X₂); and,
f) optionally, recovering methanol from said methanol (MeOH) stream and/or recovering dimethyl ether (DME) from said methanol (MeOH) stream.

In certain embodiments, a process for producing methanol (MeOH) and hydrogen from methane according to an embodiment of the disclosure, comprises the steps of:
a) providing a gaseous feed stream comprising methane;
b) reacting said gaseous feed stream with at least one halogen reactant (X₂), thereby producing an effluent stream comprising hydrogen halide (HX), methyl halide (MeX), polyhalogenated alkanes, such as polyhalogenated methanes, as defined herein; optionally unreacted methane,
c) separating from the effluent stream obtained in step b):
   c1) a mono-halide stream, comprising methyl halide (MeX) and hydrogen halide (HX), optionally comprising unreacted methane; and,
   c2) a polyhalogenated alkanes stream, comprising polyhalogenated methane;
d) reacting the mono-halide stream separated in step c), with water and at least one catalyst, under reaction conditions effective to produce:
   d1) a stream of an aqueous solution of hydrogen halide (HX_{(aq)}), and
   d2) a methanol stream comprising methanol (MeOH), and optionally dimethyl ether (DME), and/or optionally unreacted methane,
e) decomposing by means of electrolysis said aqueous solution of hydrogen halide (HX_{(aq)}) produced in step d1) under conditions effective to produce a gaseous hydrogen (H₂) stream and a stream comprising halogen reactant (X₂); and,
f) optionally, recovering methanol from said methanol (MeOH) stream;
g) optionally, recovering at least a part of said at least one catalyst after step d) or during step e) of the process, and supplying said recovered catalyst in step d) of the process; and,
h) optionally, returning the halogen reactant (X₂) obtained in step e), to step b) of the process.

For example, a process for producing methanol (MeOH), dimethyl ether (DME) and hydrogen from methane according to an embodiment of the disclosure, comprises the steps of:
a) providing a gaseous feed stream comprising methane;
b) reacting said gaseous feed stream with at least one halogen reactant (X₂), thereby producing an effluent stream comprising hydrogen halide (HX), methyl halide (MeX), polyhalogenated alkanes, such as polyhalogenated methanes, as defined herein; optionally unreacted methane,
c) separating from the effluent stream obtained in step b):
   c1) a mono-halide stream, comprising methyl halide (MeX) and hydrogen halide (HX), optionally comprising unreacted methane; and,
   c2) a polyhalogenated alkanes stream, comprising polyhalogenated methane;
d) reacting the mono-halide stream separated in step c), with water and an organic base, under reaction conditions effective to produce:
   d1) a stream of an aqueous solution of hydrogen halide (HX_{(aq)}), and
   d2) a methanol stream comprising methanol (MeOH), and dimethyl ether (DME), and optionally unreacted methane,
e) decomposing by means of electrolysis said aqueous solution of hydrogen halide (HX_{(aq)}) produced in step d1) under conditions effective to produce a gaseous hydrogen (H₂) stream and a stream comprising halogen reactant (X₂); and,
f) optionally, recovering methanol from said methanol (MeOH) stream and/or recovering dimethyl ether (DME) from said methanol (MeOH) stream;
g) optionally, recovering at least a part of said organic base after step d) or during step
e) of the process, and supplying said recovered organic base in step d) of the process; and,
h) optionally, returning the halogen reactant (X₂) obtained in step e), to step b) of the process.

It is preferred according to the disclosure that steps b) and d) are performed in separate reaction steps, preferably in separate reactors or separate reaction zones. The process of the disclosure is preferably a continuous process.

The present disclosure provides a process for producing a stream of hydrogen and a stream of methanol and/or dimethyl ether, preferably a stream of hydrogen and a stream of methanol and dimethyl ether, starting from a gaseous feed stream which comprises or essentially consists of methane, such as a biogas or a natural gas. In other words, the present disclosure provides a process for producing methanol and/or dimethyl ether, preferably methanol and dimethyl ether, from a gaseous stream comprising methane which is not syngas.

The present process comprises the reaction of the gaseous feed stream with at least one halogen reactant to form methyl halide and hydrogen halide, followed by a reaction of the produced methyl halide with water and optionally at least one catalyst which is preferably an organic base thereby forming methanol and/or dimethyl ether, preferably methanol and dimethyl ether. The present process further produces an aqueous solution of hydrogen halide, which may be treated electrolytically to be decomposed in separate streams of hydrogen and halogen reactant, yielding a valuable side product, i.e., hydrogen. Moreover, the recovered halogen reactant may advantageously be re-used in the present process.

The present process thus provides an efficient process for the conversion of methane in methanol and/or dimethyl ether, preferably in methanol and dimethyl ether, with the production of hydrogen which is an industrially relevant side product.

A first step of the process comprises the provision of a gaseous feed stream comprising methane.

The term “gaseous feed stream” and “reaction gas” are used herein as synonyms and refers to a gaseous stream comprising methane.

In certain embodiments of a process according to the disclosure, the applied gaseous feed stream may also comprise C₂₊ alkanes.

The term “alkane” refers to an organic compound being either an acyclic saturated hydrocarbon or a cyclic saturated hydrocarbon. As used herein alkanes might be straight, branched or cyclic (but not aromatic). Preferably, the alkanes are straight or branched. In preferred embodiments, the alkanes are acyclic saturated hydrocarbons and fulfil the following formula CᵢH₍₂ᵢ₊₂₎, wherein i is an integer. Examples are methane, ethane, propane, butane, pentane and hexane.

The term “C₂₊ alkanes” as used herein intends to refer to alkanes having more than two carbon atoms. In some embodiments, the C₂₊ alkanes are C₂₋₆ alkanes, i.e., alkanes comprising two to six carbon atoms, thus for instance C₂, C₃, C₄, C₅, C₆ alkanes, and any mixtures thereof. When a subscript is used herein following a carbon atom, the subscript refers to the number of carbon atoms that the named group may contain. Examples of C₂₊ alkanes are ethane, propane, butanes, pentanes, the hexanes, and mixtures of two or more of these.

For instance, in certain embodiments, the applied gaseous feed stream may comprise a mixture of methane and ethane, or a mixture of methane and propane, or a mixture of methane, ethane, and propane.

Preferably, a gaseous feed stream comprising methane for use in the present process, comprises at least 75.0 vol. % of methane, preferably at least 80.0 vol. % of methane, preferably at least 85.0 vol. % of methane, preferably at least 90.0 vol. % of methane, preferably at least 95.0 vol. % of methane, preferably at least 98.0 vol. % of methane, preferably at least 99.0 vol. % of methane, preferably at least 99.9 vol. % of methane, based on the total volume of the gaseous feed stream.

Optionally, a gaseous feed stream as applied in a process according to the disclosure may also comprise hydrogen. Preferably, the molar ratio of this optional hydrogen to methane in said gaseous feed stream may be in the range from about 1:4 to 0:1.

Optionally, a gaseous feed stream as applied in a process according to the disclosure may also comprise minor amounts of other components selected from oxygen, nitrogen, or carbon dioxide. Preferably such other component may be present in an amount lower than 2.0 mol. %, such as lower than 1.5 mol. %, or lower than 1.0 mol. % or lower than 0.5 mol. %, based on the total molar content of said gaseous feed stream. In certain embodiments, the composition of the gaseous feed stream is comparable to the composition of a natural gas, or a biogas, or a mixture thereof.

In certain embodiments, the gaseous feed stream applied in step a) of the process comprises a natural gas, a biogas; a refinery gas (i.e., the gas fraction coming from the refinery of crude oil) or any mixtures thereof. Preferably, the gaseous feed stream comprises natural gas and/or biogas.

In certain embodiments of a process according to the disclosure, the gaseous feed stream applied in step a) of the process gas is a natural gas. The term “natural gas” refers to a multi-component gas obtained from a crude oil well (associated gas) or from a subterranean gas-bearing formation (non-associated gas). The composition and pressure of natural gas can vary significantly. A typical natural gas stream contains methane as a significant component. Natural gas may also contain ethane, higher molecular weight hydrocarbons, such as C₂₊ alkanes, acid gases (such as carbon dioxide, hydrogen sulphide, carbonyl sulphide, carbon disulphide, and mercaptans), and minor amounts of contaminants such as water, nitrogen, iron sulphide, wax, and crude oil. As used herein, “natural gas” may also include gas resulting from the regasification of a liquefied natural gas, which has been purified to remove contaminates, such as water, acid gases, and most of the higher molecular weight hydrocarbons (e.g., C₁₂+ hydrocarbons). Conventional methods can be used for removing impurities and/or adjusting the relative amount of hydrocarbon compounds present in the gaseous feed stream.

In certain embodiments of a process according to the disclosure, the gaseous feed stream applied in step a) of the process gas is a biogas or a biomethane produced from biogas. The term “biogas” refers to a multi-component gas, primarily consisting of methane and carbon dioxide, produced from anaerobic digestion or by methanogenesis from raw materials such as but not limited to agricultural waste, manure, municipal waste, plant material, sewage, green waste or food waste. Biogas may be purified to remove carbon dioxide, oxygen, sulphur, and/or silicon-containing compounds to produce biomethane, which could be applied as gaseous feed stream in the present process.

In certain embodiments, said natural gas may be of fossil origin. In certain embodiments, said natural gas may be of renewable origin. Natural gas of renewable origin for instance includes gas produced from existing waste streams and a variety of renewable and sustainable biomass sources, including but not limited to animal waste, crop residuals and food waste, organic waste from dairies and farm, and naturally-occurring biological breakdown of organic waste at facilities such as wastewater treatment plants and landfills. In certain embodiments, said natural gas may comprise a combination of natural gas from fossil origin and from renewable source.

Preferably, the gaseous feed stream applied in step a) of the process is substantially free of sulphur species, e.g., it contains less than 1.0 mol. %, or less than 0.5 mol. %, or less than 0.1 mol. %, or less than 0.01 mol. %, or less than 0.001 mol. %, or less than 0.0001 mol. %, or less than 0.00001 mol. % of sulphur species based on the total molar content of said gaseous feed stream. In an example, the gaseous feed stream is free of sulphur species. Sulphur species may for instance refer to species in the form of H₂S, COS, CS₂. Desulphurisation, if necessary, can be achieved by adsorption and/or absorption.

In certain other embodiments of a process according to the disclosure, the gaseous feed stream consists of methane.

Step b) of a process of the disclosure comprises reacting the gaseous feed stream as defined herein with at least one halogen reactant (X₂), under reaction conditions effective to produce an effluent stream comprising methyl halide (MeX), hydrogen halide (HX) and optionally polyhalogenated alkanes. The effluent stream may optionally comprise unreacted methane.

The halogen reactant applied in this step of the present process may be selected from the group consisting of bromine (Br₂), chlorine (Cl₂), fluorine (F₂), and iodine (I₂), and preferably is bromine. Especially, the recovery of bromine in the process e) and f) requires less energy, reagents, and/or equipment.

Therefore, in a preferred embodiment, the disclosure provides a process for producing methanol (MeOH) and hydrogen (H₂) from methane, comprising the steps of:
a) providing a gaseous feed stream comprising methane,
b) reacting said gaseous feed stream with bromide (Br₂), thereby producing an effluent stream comprising hydrogen bromide (HBr), methyl bromide (MeBr), optionally polybrominated alkanes, such as polybrominated methanes; and optionally unreacted methane,
c) optionally, separating from the effluent stream obtained in step b):
   (i) a mono-bromide stream, comprising methyl bromide (MeBr) and hydrogen bromide (HBr), optionally comprising unreacted methane; and,
   (ii) a polybrominated alkanes stream, comprising polybrominated methanes;
d) reacting the effluent stream obtained in step b), or the mono-bromide stream separated in step c), with water and at least one catalyst, under reaction conditions effective to produce:
   d1) a stream of an aqueous solution of hydrogen bromide (HBr_{(aq)}), and
   d2) a methanol stream comprising methanol (MeOH), and optionally dimethyl ether (DME), and/or optionally unreacted methane,
e) decomposing by means of electrolysis said aqueous solution of hydrogen bromide (HBr_{(aq)}) obtained in step d1) under conditions effective to produce a gaseous hydrogen (H₂) stream and a stream comprising bromine (Br₂);
f) optionally recovering methanol from said methanol (MeOH) stream; and,
g) optionally, recovering at least a part of said at least one catalyst after step d) or during step e) of the process, and supplying said recovered catalyst in step d) of the process; and,
h) optionally, returning bromine (Br₂) obtained in step e), to step b) of the process.

For example, the disclosure provides a process for producing methanol (MeOH), dimethyl ether (DME) and hydrogen (H₂) from methane, comprising the steps of:
a) providing a gaseous feed stream comprising methane,
b) reacting said gaseous feed stream with bromide (Br₂), thereby producing an effluent stream comprising hydrogen bromide (HBr), methyl bromide (MeBr), optionally polybrominated alkanes, such as polybrominated methanes; and optionally unreacted methane,
c) optionally, separating from the effluent stream obtained in step b):
   (i) a mono-bromide stream, comprising methyl bromide (MeBr) and hydrogen bromide (HBr), optionally comprising unreacted methane; and,
   (ii) a polybrominated alkanes stream, comprising polybrominated methanes;
d) reacting the effluent stream obtained in step b), or the mono-bromide stream separated in step c), with water and an organic base, under reaction conditions effective to produce:
   d1) a stream of an aqueous solution of hydrogen bromide (HBr_{(aq)}), and
   d2) a methanol stream comprising methanol (MeOH), and dimethyl ether (DME), and optionally unreacted methane,
e) decomposing by means of electrolysis said aqueous solution of hydrogen bromide (HBr_{(aq)}) obtained in step d1) under conditions effective to produce a gaseous hydrogen (H₂) stream and a stream comprising bromine (Br₂);
f) optionally recovering methanol from said methanol (MeOH) stream and/or recovering dimethyl ether (DME) from said methanol (MeOH) stream; and,
g) optionally, recovering at least a part of said organic base after step d) or during step e) of the process, and supplying said recovered organic base in step d) of the process; and,
h) optionally, returning bromine (Br₂) obtained in step e), to step b) of the process.

Preferably, the such as polybrominated methanes are selected from CH₂Br₂, CHBr₃ and CBr₄ and combinations thereof.

Preferably, the halogen reactant, such as bromine, is introduced directly into the reaction zone. The halogen reactant may be introduced in the reaction zone in liquid state. The halogen reactant may be introduced in the reaction zone in pure form, or dissolved in an aqueous hydrogen halide solution.

In accordance with the present process, step b) of the process is preferably carried at a temperature during step b) of at least 150° C., preferably at least 200° C., preferably at least 250° C., preferably at least 300° C., preferably at least 350° C., preferably at least 380° C. In accordance with the present process, step b) of the process is preferably carried at a temperature during step b) of at most 900° C., preferably at most 800° C., preferably at most 700° C., preferably at most 600° C., preferably at most 550° C., preferably at most 520° C.

For instance, preferred temperature conditions for the reaction between the feed stream comprising methane and the halogen reactant in the present process are comprised between 150° C. and 900° C., preferably comprised between 200° C. and 800° C., preferably comprised between 250° C. and 700° C., preferably comprised between 300° C. and 600° C., preferably comprised between 350° C. and 550° C., preferably comprised between 380° C. and 520° C. In some embodiments, the exothermic nature of the reaction is sufficient to maintain the temperature during the reaction.

In accordance with the present process, step b) of the process is carried out at a reaction pressure of at least 1.0 bar, preferably at least 1.5 bar, preferably at least 2.0 bar, preferably at least 2.5 bar, preferably at least 3.0 bar, preferably at least 5.0 bar, preferably at least 10.0 bar. In accordance with the present process, step b) of the process is carried out at a reaction pressure of at most 50.0 bar, preferably at most 45.0 bar, preferably at most 40.0 bar, preferably at most 35.0 bar, preferably at most 30.0 bar, preferably at most 25.0 bar, preferably at most 20.0 bar.

For instance, preferred pressure conditions for the reaction between the feed stream comprising methane and the halogen reactant in the present process are comprised between 1.0 bar and 50.0 bar, preferably comprised between 1.5 bar and 45.0 bar, preferably comprised between 2.0 bar and 40.0 bar, preferably comprised between 2.5 bar and 35.0 bar, preferably comprised between 3.0 bar and 30.0 bar, preferably comprised between 5.0 bar and 25.0 bar, preferably comprised between 10.0 bar and 20.0 bar.

In certain embodiments of the present process the feed stream comprising methane and the halogen reactant are applied at a molar ratio of methane/halogen comprised between 2/1 and 7/1, preferably between 3/1 to 6/1, preferably between 4/1 to 5/1.

The time required for the gaseous feed stream and halogen to react will vary depending on the specific feed material utilized, the halogen utilized, and the temperature of the reaction. In an example, the gaseous feed stream is contacted in step b) with the halogen reactant (X₂), at a temperature of between 380° C. and 520° C., at a pressure of between 10 bar and 20 bar, and for at least 0.1 min to at most 2.0 min.

The effluent stream resulting from reaction step b) mainly comprises methyl halide and hydrogen halide. This effluent stream may also comprise certain amounts of unreacted methane. Also, depending on the composition of the initial gaseous feed stream applied in the process, the effluent stream may comprise certain amounts of C₂₊ alkyl monohalides and/or certain amounts of polyhalogenated alkanes such as polyhalogenated methane and/or polyhalogenated C₂₊ alkanes, that have been formed during reaction step b).

In a next step, the process of the disclosure comprises the step of separating from the effluent stream obtained in step b): (i) a mono-halide stream, comprising methyl halide (MeX) and hydrogen halide (HX), optionally comprising unreacted methane; and, (ii) a polyhalogenated alkanes stream, comprising polyhalogenated alkanes, such as polyhalogenated methane and optionally polyhalogenated C₂₊ alkanes if the gaseous feed stream comprises C₂₊ hydrocarbons.

The mono-halide stream is then further reacted with water and at least one catalyst which is preferably an organic base (in step d) of the process, while the aqueous hydrogen halide stream is electrolysed (in step e) of the process.

As used herein, the term “mono-halide” refers to a compound comprising a single halogen atom. The term “mono-halide” preferably covers both organic compounds and inorganic compounds. An example of an organic mono-halide is an alkyl monohalide comprising a single halogen atom, such as methyl halide (MeX). An example of an inorganic mono-halide is hydrogen halide (HX). Preferably the term “mono-halide” as used herein refers to compounds, either being methyl halide (MeX) or hydrogen halide (HX).

Therefore, as used herein, the term “mono-halide stream” refers to a stream comprising one or more mono-halides, preferably two or more mono-halides. Other non-halogenated compounds may be present in the mono-halide stream, for example, compounds such as methane. Preferably, the term “mono-halide stream” refers to a stream comprising at least methyl halide (MeX) and hydrogen halide (HX); even more preferably, the term “mono-halide stream” refers to a stream comprising, besides non-halogenated compounds, only methyl halide (MeX) and hydrogen halide (HX), as mono-halogenated compounds. Preferably, the mono-halide stream is essentially free of poly-halogenated compounds.

In preferred embodiments, the mono-halide stream is separated from the effluent stream in step c) by distillation.

In preferred embodiments, the polyhalogenated alkanes stream is separated from the effluent stream in step c) by destination.

In preferred embodiments, the effluent stream is separated into a mono-halide stream and a polyhalogenated alkanes stream in a separation column or a distillation column.

In certain embodiments of a process of the disclosure, depending on the composition of the initial gaseous feed stream applied in the process, the effluent stream may comprise additional components besides the methyl halide, the hydrogen halide and optional unreacted methane.

In certain embodiments of a process of the disclosure, polyhalogenated alkanes are formed in step b) and are present in the effluent stream obtained after step b).

The term “polyhalogenated alkanes” as used herein refers to alkanes wherein more than one hydrogen atom is replaced by a halogen atom (X), wherein X is preferably selected from the group consisting of Br, Cl, F, I, and At, more preferably X is Br. “Polyhalogenated alkanes” as used herein preferably refer to polyhalogenated alkanes such as polyhalogenated C₁-C₆ alkanes, preferably polyhalogenated C₁-C₄ alkanes, preferably polyhalogenated C₁-C₃ alkanes. When a subscript is used herein following a carbon atom, the subscript refers to the number of carbon atoms that the named group may contain.

In certain embodiments, the term “polyhalogenated alkane” refers to a compound with the following formula (I):

CₙH₍₂ₙ₊₂₎₋ₘ₎Xₘ (I)
wherein:
   n is an integer and n≥1, preferably 1≤n≥6; preferably 1≤n≥4; preferably 1≤n≥3;
   m is an integer and m≥2; preferably 2≤m≥4, preferably 2≤m≥3; and,
   X is selected from the group consisting of Br, Cl, F, I and At, preferably X is Br.

In certain embodiments, polyhalogenated alkanes comprise polyhalogenated C₁ alkanes.

In certain embodiments, polyhalogenated alkanes comprise polyhalogenated C₂₊ alkanes.

In certain embodiments, polyhalogenated alkanes comprise polyhalogenated C₁ alkanes and polyhalogenated C₂₊ alkanes.

The terms “polyhalogenated C₁ alkane” and “polyhalogenated methane” are used herein interchangeably and intend to refer to a C₁ alkane wherein more than one hydrogen atom is replaced by a halogen atom (X), wherein X is preferably selected from the group consisting of Br, Cl, F, I and At, more preferably wherein X is Br.

In certain embodiments, the terms “polyhalogenated C₁ alkane” and “polyhalogenated methane” refer to a compound with the following formula (II):

C₁H₍₄₋ₚ₎Xₚ (II)
wherein:
   p is an integer and p≥2; preferably 2≤p≥4, preferably 2≤p≥3; and,
   X is selected from the group consisting of Br, Cl, F, I and At, and preferably X is Br.

In certain preferred embodiments, a “polyhalogenated methane” is selected from the group comprising CH₂X₂, CHX₂, and CX₄, and any mixtures thereof, wherein X is as defined herein above. For instance, polyhalogenated methanes formed in step b) of the process comprise methylene dihalides (CH₂X₂). Preferred examples of polyhalogenated methanes include but are not limited to CH₂Br₂, CHBr₃, and CBr₄.

The term “polyhalogenated C₂₊ alkanes” as used herein refers to C₂₊ alkanes wherein more than one hydrogen atom is replaced by a halogen atom (X), wherein X is preferably selected from the group consisting of Br, Cl, F, I, and At, more preferably wherein X is Br. Preferably, polyhalogenated C₂₊ alkanes preferably refer to polyhalogenated C₂₊ alkanes such as polyhalogenated C₂-C₆ alkanes, preferably polyhalogenated C₂-C₄ alkanes, preferably polyhalogenated C₂-C₃ alkanes.

In certain embodiments, a “polyhalogenated C₂₊ alkane” refers to a compound with the following formula (III):

C_{q}H_{(2q+2)-f)}X_{f} (III)
wherein:
   q is an integer and q≥2, preferably 2≤q≥6; preferably 2≤q≥4; preferably 2≤q≥3;
   r is an integer and r≥2; preferably 2≤r≥4, preferably 2≤r≥3; and,
   X is selected from the group consisting of Br, Cl, F, I and At, preferably X is Br.

For instance, polyhalogenated C₂₊ alkanes formed in step b) of the process may include a polyhalogenated C₂ alkane (such as e.g., dibromoethane) and/or a polyhalogenated C₃ alkane (such as e.g., dibromopropane).

In certain embodiments of a process of the disclosure, C₂₊ alkyl monohalides are also formed in step b) and are present in the effluent stream obtained after step b).

The terms “C₂₊ alkyl monohalides” and “monohalogenated C₂₊ alkanes” are used herein interchangeably and refer to C₂₊ alkanes wherein a single hydrogen atom is replaced by a halogen atom (X), wherein X is preferably selected from the group consisting of Br, Cl, F, I and At, more preferably wherein X is Br. Preferably, the monohalogenated C₂₊ alkanes preferably refer to monohalogenated C₂₊ such as monohalogenated C₂-C₆ alkanes, preferably monohalogenated C₂-C₄ alkanes, preferably monohalogenated C₂-C₃ alkanes.

In other words, the terms “C₂₊ alkyl monohalide” and “monohalogenated C₂₊ alkane” refer to a compound with the following formula (IV):

CₛH₍₂ₛ₊₁₎X₁ (IV)
wherein:
   s is an integer and s≥2; preferably 2≤s≥6; preferably 2≤s≥4; preferably 2≤s≥3 and,
   X is selected from the group consisting of Br, Cl, F, I and At, preferably X is Br.

For instance, C₂₊ alkyl monohalides formed in step b) of the process may include a C₂ alkyl monohalide (e.g., ethyl bromide) and/or a C₃ alkyl monohalide (such as e.g., n-propyl bromide).

The present process may comprise a separation of said polyhalogenated alkanes and/or C₂₊ alkyl monohalides from said effluent gas. Therefore, in certain embodiments, step c) in a process of the disclosure comprises the step of separating from the effluent stream obtained in step b):
(i) a mono-halide stream, comprising methyl halide (MeX) and hydrogen halide (HX), optionally comprising unreacted methane;
(ii) a polyhalogenated alkanes stream, comprising polyhalogenated alkanes as defined herein; and,
(iii) optionally a C₂₊ alkyl monohalide stream, comprising C₂₊ alkyl monohalides as defined herein.

In some embodiments, step c) comprises a single separation step of the effluent stream, i.e., the effluent stream is separated in one step. In some embodiments, step c) comprises two separation steps, i.e., a mono-halide separation step and a polyhalogenated alkanes separation step. In some embodiments, step c) comprises three separation steps, i.e., a mono halide separation step, a polyhalogenated alkanes separation step, and a C₂₊ alkyl monohalide separation step.

In one example, step c) in a process of the disclosure comprises separating from the effluent stream obtained in step b):
a mono-halide stream, comprising methyl halide (MeX) and hydrogen halide (HX), optionally comprising unreacted methane;
a polyhalogenated alkanes stream, comprising polyhalogenated alkanes as defined herein; and,
optionally a C₂₊ alkyl monohalide stream, comprising C₂₊ alkyl monohalides as defined herein.

In another example, step c) in a process of the disclosure comprises separating from the effluent stream obtained in step b):
a mono-halide stream, comprising methyl halide (MeX) and hydrogen halide (HX);
a polyhalogenated alkanes stream, comprising polyhalogenated alkanes as defined herein;
a C₂₊ alkyl monohalide stream; and,
an unreacted methane stream.

In another example, step c) in a process of the disclosure comprises separating from the effluent stream obtained in step b):
a mono-halide stream, comprising methyl halide (MeX) and hydrogen halide (HX), optionally comprising unreacted methane;
a polyhalogenated methane stream comprising polyhalogenated methanes as defined herein;
a polyhalogenated C₂₊ alkanes stream; comprising polyhalogenated C₂₊ alkanes as defined herein; and,
a C₂₊ alkyl monohalide stream, comprising C₂₊ alkyl monohalides as defined herein.

Separation of polyhalogenated alkanes, and C₂₊ alkyl monohalides from the effluent stream has the benefit of allowing to recover a purified mono-halide stream, comprising methyl halide (MeX) and hydrogen halide, which contains less or no other polyhalogenated alkanes such as polyhalogenated methane and/or polyhalogenated C₂₊ alkanes, and hence such purified mono-halide stream can advantageously be converted into a stream of methanol and/or dimethyl ether, preferably a stream of methanol and dimethyl ether, without the generation of significant amounts of non-alcohol products apart from alkanes and DME. This way, a methanol can be produces with less than 10.000 ppm, preferably less than 7.000 ppm, preferably less than 5.000 ppm, preferably less than 3.000 ppm, preferably less than 2.000 ppm, preferably less than 1.000 ppm non-alcoholic products apart from alkanes and DME.

In particularly, a preferred process for producing methanol (MeOH) and hydrogen (H₂) from methane according to the disclosure, comprising the steps of:
a) providing a gaseous feed stream comprising methane;
b) reacting said gaseous feed stream with at least one halogen reactant (X₂), under reaction conditions effective to produce an effluent stream comprising: hydrogen halide (HX), methyl halide (MeX), polyhalogenated alkanes, such as polyhalogenated methanes as defined herein, and, optionally unreacted methane,
c) separating from the effluent stream obtained in step b):
   c1) a mono-halide stream, comprising methyl halide (MeX) and hydrogen halide (HX), optionally comprising unreacted methane; and,
   c2) a polyhalogenated alkanes stream, comprising polyhalogenated methanes as defined herein;
   c3) a C₂₊ alkyl monohalide stream comprising C₂₊ alkyl monohalides as defined herein;
d) reacting the mono-halide stream separated in step c), with water and at least one catalyst, under reaction conditions effective to produce:
   d1) a stream of an aqueous solution of hydrogen halide (HX_{(aq)}), and
   d2) a methanol stream comprising methanol (MeOH), and optionally dimethyl ether (DME), and/or optionally unreacted methane,
e) decomposing by means of electrolysis said aqueous solution of hydrogen halide (HX_{(aq)}) obtained in step d1) under conditions effective to produce a gaseous hydrogen (H₂) stream and a stream comprising halogen reactant (X₂);
f) optionally, recovering methanol from said methanol (MeOH) stream;
g) optionally recovering at least a part of said at least one catalyst after step d) or during step e) of the process, and supplying said recovered catalyst in step d) of the process;
h) optionally, returning the halogen reactant (X₂) obtained in step e), to step b) of the process; and,
i) optionally, converting the polyhalogenated alkanes stream and/or the C₂₊ alkyl monohalide stream into their corresponding alcohols.

For example, a process for producing methanol (MeOH), dimethyl ether (DME) and hydrogen (H₂) from methane according to the disclosure, comprising the steps of:
a) providing a gaseous feed stream comprising methane;
b) reacting said gaseous feed stream with at least one halogen reactant (X₂), under reaction conditions effective to produce an effluent stream comprising: hydrogen halide (HX), methyl halide (MeX), polyhalogenated alkanes, such as polyhalogenated methanes as defined herein, and, optionally unreacted methane,
c) separating from the effluent stream obtained in step b):
   c1) a mono-halide stream, comprising methyl halide (MeX) and hydrogen halide (HX), optionally comprising unreacted methane; and,
   c2) a polyhalogenated alkanes stream, comprising polyhalogenated methanes as defined herein;
   c3) a C₂₊ alkyl monohalide stream comprising C₂₊ alkyl monohalides as defined herein;
d) reacting the mono-halide stream separated in step c), with water and an organic base, under reaction conditions effective to produce:
   d1) a stream of an aqueous solution of hydrogen halide (HX_{(aq)}), and
   d2) a methanol stream comprising methanol (MeOH), and dimethyl ether (DME), and optionally unreacted methane,
e) decomposing by means of electrolysis said aqueous solution of hydrogen halide (HX_{(aq)}) obtained in step d1) under conditions effective to produce a gaseous hydrogen (H₂) stream and a stream comprising halogen reactant (X₂);
f) optionally, recovering methanol from said methanol (MeOH) stream and/or recovering dimethyl ether (DME) from said methanol (MeOH) stream;
g) optionally recovering at least a part of said organic base after step d) or during step e) of the process, and supplying said recovered organic base in step d) of the process;
h) optionally, returning the halogen reactant (X₂) obtained in step e), to step b) of the process; and,
i) optionally, converting the polyhalogenated alkanes stream and/or the C₂₊ alkyl monohalide stream into their corresponding alcohols.

Preferably, polyhalogenated alkanes formed in step b) of the process are separated from the effluent stream by distillation. For instance, a polyhalogenated alkane stream or a polyhalogenated methane stream and a polyhalogenated C₂₊ alkanes stream can be separated from the effluent stream in a separation column. Preferably, as there is a large difference in molecular weight between the polyhalogenated alkanes and the mono-halogenated compounds, and thus a large difference in boiling point, distillation may be a preferred separation technique. Preferably polyhalogenated alkanes are separated from the effluent stream before the mono-halide stream, i.e., methyl halide, hydrogen halide and optionally C₂₊ alkyl monohalides, are separated from the effluent stream.

In certain preferred embodiments of the present disclosure, the process comprises the step of separating polyhalogenated alkanes formed in step b) from said effluent stream, thereby creating a polyhalogenated alkanes stream, whereby said polyhalogenated alkanes formed in step b) are separated from said effluent stream prior to separating the mono-halide stream from said effluent stream.

Preferably, C₂₊ alkyl monohalides formed in step b) of the process are separated from the effluent stream by distillation. For instance, a C₂₊ alkyl monohalide stream can be separated from the effluent stream in a separation column.

In certain embodiments, the present process comprises the separation of polyhalogenated methane stream and polyhalogenated C₂₊ alkanes stream in separate steps and/or in separate separation columns. Alternatively, the present process may comprise the separation of polyhalogenated methane stream and polyhalogenated C₂₊ alkanes stream in a single step and/or in a single separation column.

In certain preferred embodiments of the present disclosure, the process comprises the step of separating C₂₊ alkyl monohalides formed in step b) of the process from said effluent stream, thereby creating a C₂₊ alkyl monohalide stream, whereby said C₂₊ alkyl monohalides formed in step b) are separated from said effluent stream after separating the polyhalogenated alkanes stream from said effluent stream, preferably by distillation.

In certain embodiments, the present process comprises the separation of polyhalogenated alkanes stream and mono-halide stream in separate steps and/or in separate separation columns. Alternatively, the present process may comprise the separation of polyhalogenated alkanes stream and mono-halide stream in a single step and/or in a single separation column.

The mono-halide stream separated in step c) is preferably a gaseous stream.

Upon separating the polyhalogenated alkane and/or C₂₊ alkyl monohalide streams, from said effluent stream in step c), the present process allows to separate a mono-halide stream which mainly comprises methyl halide (MeX) and hydrogen halide (HX). Preferably the separated mono-halide stream comprises at least 75.0 mol. %, such as least 80.0 mol. %, or at least 85.0 mol. %, or at least 90.0 mol. % of mono-halide, based of the total molar content of said separated mono-halide stream. It will be noted however that the separated mono-halide stream may still comprise certain amounts of unreacted methane.

In accordance with the present disclosure, the mono-halide stream separated in step c), is also preferably “essentially free” of polyhalogenated alkanes. “Essentially free of polyhalogenated alkanes” in this context means that the mono-halide stream obtained in step c), comprises less than 1.0 mol. %, such as less than 0.1 mol. %, preferably less than 0.5 mol. % of polyhalogenated alkanes, as defined herein, based on the total molar content of said mono-halide stream obtained in step c).

In accordance with the present disclosure, the recovered mono-halide stream obtained in step c), is also preferably “essentially free” of C₂₊ alkyl monohalides. Preferably, “essentially free of C₂₊ alkyl monohalides” in this context means that the mono-halide stream obtained in step c), comprises less than 1.0 mol. %, such as less than 0.1 mol. % preferably less than 0.5 mol. % C₂₊ alkyl monohalides, as defined herein, based on the total molar content of the mono-halide stream obtained in step c).

In a next step, the mono-halide stream separated in step c) of the process is reacted with water and at least one catalyst which is preferably an organic base, under reaction conditions effective to produce an aqueous solution of hydrogen halide (HX_{(aq)}), and methanol (MeOH), or preferably an aqueous solution of hydrogen halide (HX_{(aq)}), methanol (MeOH) and dimethyl ether (DME).

Step d) of the process of the disclosure is preferably carried at a temperature of at least 50° C., preferably at least 70° C., preferably at least 80° C., preferably at least 90° C., preferably at least 100° C., preferably at least 150° C. Step d) of the process of the disclosure is preferably carried out at a temperature of at most 500° C., preferably at most 450° C., preferably at most 400° C., preferably at most 350° C., preferably at most 300° C., preferably at most 250° C. For instance, preferred temperature conditions for the reaction step d) are comprised between 50° C. and 500° C., preferably between 70° C. and 450° C., preferably between 80° C. and 400° C., preferably between 90° C. and 350° C., preferably between 100° C. and 300° C., preferably between 120° C. and 250° C. In some embodiments, the temperature in step d) can be achieved by heat integration, for example with heat recovered during the exothermic reaction in step b).

Step d) of the process of the disclosure is preferably carried at a reaction pression of at least 1.0 bar, preferably at least 1.2 bar, preferably at least 1.5 bar, preferably at least 1.7 bar, preferably at least 2.0 bar, preferably at least 5.0 bar, preferably at least 10.0 bar. Step d) of the process of the disclosure is preferably carried at a reaction pression of at most 40.0 bar, preferably at most 35.0 bar, preferably at most 30.0 bar, preferably at most 25.0 bar, preferably at most 20.0 bar, preferably at most 15.0 bar, preferably at most 10.0 bar. For instance, preferred pressure conditions for the reaction step d) are comprised between 1.0 bar and 40.0 bar, preferably between 1.2 bar and 35.0 bar, preferably between 1.5 bar and 30.0 bar, preferably between 1.7 bar and 25.0 bar, preferably between 2.0 bar and 20.0 bar, preferably between 5.0 bar and 20.0 bar, preferably between 10.0 bar and 15.0 bar.

Preferably, the process of the disclosure, comprises a reaction in step d) of the mono-halide stream with water and at least one catalyst which is preferably an organic base, for at least 0.1 min to at most 600.0 min, preferably for at least 0.2 min to at most 400.0 min, preferably for at least 0.5 min to at most 200.0 min, preferably for at least 1.0 min to at most 100.0 min, preferably for at least 2.0 min to at most 75.0 min, preferably for at least 3.0 min to at most 60.0 min, preferably for at least 4.0 min to at most 30.0 min, preferably for at least 5.0 min to at most 15.0 min.

In an example, the mono-halide stream is contacted in step d) with water optionally comprising at least one catalyst which is preferably an organic base, at a temperature of between 50° C. and 500° C. at a pressure of between 1.0 bar and 40.0 bar, and for at least 0.1 min to at most 600.0 min.

The reaction products resulting from reaction step d) mainly comprises an aqueous hydrogen halide solution and methanol (methanol stream), preferably an aqueous hydrogen halide solution, methanol and dimethyl ether. Optionally residual amounts of unreacted methane are also present in the reaction output.

A methanol stream comprising methanol and optionally also dimethyl ether is preferably recovered after step d). Preferably said methanol stream comprising methanol, and optionally dimethyl ether (DME), obtained in step d2) of the present process, is separated from the aqueous solution of hydrogen halide by means of distillation.

Preferably, the total amount of methanol and optionally dimethyl ether in the separated methanol stream comprises, at least 75.0 mol. %, such as least 80.0 mol. %, or at least 85.0 mol. %, or at least 95.0 mol. % based on the total molar content of the separated methanol stream. It will be noted however that the recovered methanol stream may still comprise certain amounts of unreacted methane.

By separating the polyhalogenated alkanes stream from the effluent stream obtained after step b) of the process of the disclosure, the present process has the advantage of subjecting a “purified” mono-halide stream to the hydrolysis reaction in step d) in the presence of water and at least one catalyst which is preferably an organic base. The separation step applied in step c) therefore permits to avoid a reaction of polyhalogenated alkanes with said water and optionally at least one catalyst which is preferably an organic base in step d). In this way, the present process thus avoids the conversion of polyhalogenated alkanes into hydroxylated products other than methanol or dimethyl ether during step d) of the process.

In certain embodiments, a catalyst for use in the present process is a base, and preferably an organic base. The base may deprotonate the hydrogen halide during step d).

As used herein, the term “organic base” refers to a non-metallic and basic organic species.

The organic base may contain an amino group, a pyridinyl group, a derivative of a carboxylic acid, or mixtures thereof. Non-limiting examples include for instance pyridine, alkyl amine, morpholine, imidazole, benzimidazole, triethylamine, tri-ethylenediamine, benzyldiethylamine, dimethylethyl amine, di-isopropyl ethylamine (DIPEA), 1,8-diazabicyclo[5.4.0]undec-7-ene (DBU), 2,6-dimethylpyridine (2,6-lutidine), piperidine, salts of carboxylic acids, or mixtures thereof. In a preferred embodiment, a catalyst for use in the present process is pyridine

In certain preferred embodiments, a catalyst for use in the present process is an N-containing polymer (or polymeric compound). Preferably such N-containing polymer is a polymer comprising at least one amino group, pyridine ring, piperidine ring, pyridazine ring, pyrimidine ring, pyrazine ring, triazine ring, pyrazole ring, imidazole ring, oxazole ring, isoxazole ring, thiazole ring, isothiazole ring, triazole ring, oxadiazole ring, or any combinations thereof. For example, the N-containing polymer is a polymer comprising at least one pyridine ring.

More preferably, in certain embodiments, a catalyst for use in the present process is a pyridine-containing polymer, such as for instance selected from the group comprising poly(4-vinylpyridine), poly(2-vinylpyridine), poly[2-(4-vinylphenyl)pyridine], poly[5-vinyl-2,2′-bipyridine], poly[3-(4-vinylphenyl)pyridine] and poly[4-(4-vinylphenyl)pyridine]. In some preferred examples, a catalyst for use in the present process is selected from poly(4-vinylpyridine) or poly[4-(4-vinylphenyl)pyridine].

In certain embodiments, it is preferred that a catalyst as applied in the present process is soluble in water. For instance, it may be used at a concentration of at least 5 wt. %, such as 10 wt. %, or at least 15 wt. % of an aqueous solution of said catalyst.

In some embodiments, a catalyst for use in a process of the disclosure is in a solid form or is immobilised on a solid support. In some embodiments, a solid catalyst or an immobilised catalyst is suspended in the water. In some embodiments, a solid catalyst or an immobilised catalyst used in the present process may be packed in a bed or attached to structures within a reactor.

One of ordinary skill in the art will be able to select a suitable catalyst for use in the present process without undue experimentation.

The aqueous solution of hydrogen halide (HX_{(aq)}) obtained in step d) of the process is subject to decomposition to release the hydrogen for recovery and halogen. Said halogen reactant may be recycled to step a) and/or b) of the process, for instance added to the feed stream supplied in step a) and/or separately supplied during reaction step b).

The preferred mode of hydrogen halide decomposition is electrolytic. Thus, this step of a process of the disclosure preferably takes place in an electrolysis cell, preferably in a polymer electrolyte membrane cell (PEM) containing at least a proton-conductive membrane. Polymer electrolyte membrane (PEM) electrolysis is well known in the art an comprises a cell equipped with a solid polymer electrolyte (SPE) that is responsible for the conduction of protons, separation of product gases, and electrical insulation of the electrodes.

In some embodiments of the disclosure, step e) of the present process comprises the step of supplying an aqueous solution of hydrogen halide to an electrolysis cell (as defined herein) containing positive and negative electrodes, and decomposing said hydrogen halide electrolytically by maintaining an electrical potential from about 0.5 to 2.5 V between said electrodes.

In some embodiments of the disclosure, step e) of the present process comprises the step of supplying an aqueous solution of hydrogen halide to an electrolysis cell (as defined herein) containing positive and negative electrodes, and decomposing said hydrogen halide electrolytically by maintaining a current density from about 100 to 800 mA/cm² between said electrodes.

The electrical potential required to decompose the hydrogen halide solution decreases as the temperature of the aqueous solution increases. It is particularly preferred to practice the electrolytic decomposition at a temperature of from about 20 to 95° C., and preferably from about 40 to 80° C. The pressure in the electrolytic decomposition zone is maintained sufficiently high to maintain the aqueous hydrogen halide in a liquid phase. Generally, the pressure will be within a range from about 0.1 to 5 Mpa (1-50 bar).

The gaseous hydrogen (H₂) stream generated in step e) of the process comprises at least 90.0 mol %, such as at least 95.0 mol %, or at least 99.0 mol %, or at least 99.5 mol %, or at least 99.9 mol % hydrogen. Hydrogen obtained with a process according to the disclosure may be used in downstream applications. For instance, it may be used in ammonia synthesis, for hydrogenation purposes, for chemicals synthesis, or power generation by combustion in a gas turbine with or without additional hydrocarbon fuels, etc. Hydrogen produced can also be applied as a chemical feedstock to reduce dependence on petroleum and natural gas.

The halogen reactant (X₂) generated in step e) of the process can be re-used in step b) of the process, for instance after a suitable pre-treatment, such as a purification and/or drying.

In some preferred embodiments, the halogen reactant (X₂) obtained in step e) is purified before being returned into step b), e.g., by condensation.

In some preferred embodiments, the halogen reactant (X₂) obtained in step e) is dried before being returned into step b). Preferably, is the halogen reactant (X₂) dried with molecular sieves. Preferably, the amount of water in the halogen reactant (X₂) before the halogen reactant (X₂) is returned into step b) is at most 2000 ppm, preferably at most 1500 ppm, preferably at most 1000 ppm, preferably at most 750 ppm, preferably at most 500 ppm, wherein the ppm is expressed as the weight of water compared to the total weight of the halogen reactant.

In certain embodiments, the present process comprises the step of recovering at least a part of said at least one catalyst which is preferably an organic base after step d) or during step e) of the process, and comprises the step of supplying said recovered catalyst in step d) of the process.

In some embodiments, the aqueous solution of hydrogen halide (HX_{(aq)}) obtained in step d1) may comprise catalyst, especially when the catalyst is a water-soluble organic base, such as e.g., pyridine. In such embodiments, such catalyst may remain present after electrolysis step e). In some embodiments of the process, such catalyst, remaining after the electrolytic decomposition in step e), may be recovered and supplied in step d) and re-used in the process. In an example, in accordance with the present process, a part of the catalyst will be circulating in the hydrolysis reactor and/or in the electrolysis cell.

In some other embodiments, catalyst, preferably catalyst provided in solid or immobilised form, may be prevented from being transferred with the aqueous solution of hydrogen halide (HX_{(aq)}) to an electrolysis unit for decomposition. In an example, this can be achieved by using suitable filtering means. In such embodiments, fresh water coming into the process will allow to re-use the catalyst remaining after step d).

In certain embodiments of a process according to the disclosure, polyhalogenated methanes (as defined herein) are formed during step b), and may be separated during step c), as indicated above. In certain embodiments, a process of the disclosure therefore further comprises the step of converting said polyhalogenated methanes, e.g., separated in step c), to methanol, preferably by hydrogenation of said polyhalogenated methanes to form methyl halide, followed by contacting said methyl halide with a solid metal hydroxide (MOH₍ₛ₎), under reaction conditions effective to produce the corresponding alcohols.

In certain embodiments of a process according to the disclosure, polyhalogenated C₂₊ alkanes can also be formed during step b), and may be separated during step c), as indicated above. In certain embodiments, a process of the disclosure therefore further comprises the step of converting said polyhalogenated C₂₊ alkanes to their corresponding alcohols, preferably by hydrogenation to their corresponding C₂₊ mono-halide compound, followed by contacting said C₂₊ mono-halide compound with a solid metal hydroxide (MOH₍ₛ₎), under reaction conditions effective to produce the corresponding C₂₊ alcohols.

Examples of suitable metal hydroxides for use in the present process include but for instance metal hydroxides selected from the list comprising or consisting of potassium hydroxide (KOH), sodium hydroxide (NaOH), barium hydroxide (Ba(OH)₂), caesium hydroxide (CsOH), strontium hydroxide (Sr(OH)₂), calcium hydroxide (Ca(OH)₂), lithium hydroxide (LiOH) rubidium hydroxide (RbOH), magnesium hydroxide (Mg(OH)₂), and any mixtures thereof.

Preferred examples of a solid metal hydroxide may be alkali hydroxides, such as preferably potassium hydroxide (KOH), lithium hydroxide (LiOH), sodium hydroxide (NaOH), or any mixtures thereof. A particularly preferred example is potassium hydroxide (KOH).

The term “solid” as used herein intends to refer to a metal hydroxide provided on a solid support, such as a silica or an alumina support, more preferably a silica support. Preferably, the solid support is impregnated with the metal hydroxide. Preferably, the solid support is impregnated with a solution of the metal hydroxide, after which the impregnated solid support is dried.

In certain embodiments of the present process, unreacted methane may remain present in the effluent stream obtained in step b) of the process. In certain embodiments of the present process, unreacted methane may remain present in the mono-halide stream obtained in step c) of the process. In certain embodiments of the present process unreacted methane may remain present in the methanol stream obtained in step d) of the present process. Remaining unreacted methane may be removed from the mentioned streams, e.g., by distillation. In certain preferred embodiment of the disclosure, such removed unreacted methane can be recycled or fed into step a) and/or step b) of the present process.

The disclosure provides in a system for producing methanol and hydrogen from methane, comprising;
a halogenation reactor (1), configured to react a gaseous feed stream comprising methane with at least one halogen reactant (X₂) into an effluent stream comprising methyl halide (MeX), and hydrogen halide (HX), and optionally unreacted methane,
optionally, a polyhalogenated alkane removal unit (13), configured to receive an effluent stream from said halogenation reactor (1) and configured to separate from said effluent stream:
   (i) a polyhalogenated alkane stream, comprising polyhalogenated alkanes; and,
   (ii) a mono-halide stream, comprising methyl halide (MeX) and hydrogen halide (HX), and optionally comprising unreacted methane; and
a hydrolysis reactor (3), which is fluidly connected to said halogenation reactor (1) and configured to receive an effluent stream from said halogenation reactor (1); or, which is fluidly connected to said polyhalogenated alkane removal unit (13) and configured to receive a mono-halide stream from said polyhalogenated alkane removal unit (13);
and, wherein said hydrolysis reactor (3) is configured to react the effluent stream or the mono-halide stream, with water and at least one catalyst, under reaction conditions effective to produce (i) an aqueous solution of hydrogen halide (HX_{(aq)}), and (ii) a stream comprising methanol (MeOH) and optionally dimethyl ether (DME) and/or optionally unreacted methane; and
an electrolysis unit (4) comprising at least one electrolysis cell and a power source for supplying current to said electrolysis cell, which is fluidly connected to said hydrolysis reactor (3), and configured to receive an aqueous solution of hydrogen halide (HX_{(aq)}) and to decompose said aqueous solution of hydrogen halide (HX_{(aq)}) into a gaseous hydrogen (H₂) stream and a stream comprising halogen reactant (X₂).

For example, the disclosure also provides a system for producing methanol (MeOH), dimethyl ether (DME) and hydrogen (H₂) from methane, comprising;
a halogenation reactor (1), configured to react a gaseous feed stream comprising methane with at least one halogen reactant (X₂) into an effluent stream comprising methyl halide (MeX), and hydrogen halide (HX), and optionally unreacted methane; and,
a hydrolysis reactor (3), which is fluidly connected to said halogenation reactor (1) and configured to receive an effluent stream from said halogenation reactor (1); and, wherein said hydrolysis reactor (3) is configured to react the effluent stream with water and an organic base, under reaction conditions effective to produce a stream comprising methanol (MeOH) and dimethyl ether (DME) and optionally unreacted methane; and
an electrolysis unit (4) comprising at least one electrolysis cell and a power source for supplying current to said electrolysis cell, which is fluidly connected to said hydrolysis reactor (3), and configured to receive an aqueous solution of hydrogen halide (HX_{(aq)}) and to decompose said aqueous solution of hydrogen halide (HX_{(aq)}) into a gaseous hydrogen (H₂) stream and a stream comprising halogen reactant (X₂).

More particularly, the disclosure also provides a system for producing methanol (MeOH), dimethyl ether (DME) and hydrogen (H₂) from methane, comprising;
a halogenation reactor (1), configured to react a gaseous feed stream comprising methane with at least one halogen reactant (X₂) into an effluent stream comprising methyl halide (MeX), and hydrogen halide (HX), and optionally unreacted methane; and,
a hydrolysis reactor (3), which is fluidly connected to said halogenation reactor (1) and configured to receive an effluent stream from said halogenation reactor (1); and, wherein said hydrolysis reactor (3) is configured to react the effluent stream with water and an organic base, under reaction conditions effective to produce a stream comprising methanol (MeOH) and dimethyl ether (DME) and optionally unreacted methane; and
an electrolysis unit (4) comprising at least one electrolysis cell and a power source for supplying current to said electrolysis cell, which is fluidly connected to said hydrolysis reactor (3), and configured to receive an aqueous solution of hydrogen halide (HX_{(aq)}) and to decompose said aqueous solution of hydrogen halide (HX_{(aq)}) into a gaseous hydrogen (H₂) stream and a stream comprising halogen reactant (X₂);
a feed stream supply system (5) for supplying a gaseous feed stream comprising methane to said halogenation reactor (1);
a halogen supply system (6) for supplying a halogen reactant (X₂) to said halogenation reactor (1);
an effluent recovery system (10, 15), configured to recover an effluent stream from said halogenation reactor (1), and for feeding said recovered effluent stream to said hydrolysis reactor (3);
a methanol recovery system (7), configured to recover a stream comprising methanol (MeOH), and dimethyl ether (DME) and optionally unreacted methane from said hydrolysis reactor (3);
a hydrogen recovery system (8), configured to recover a gaseous hydrogen (H₂) stream from said electrolysis unit (4);
a halogen recovery system (9), configured to recover halogen reactant (X₂) from said electrolysis unit (4);
a hydrogen halide transfer system (12), configured to recover an aqueous solution of hydrogen halide (HX_{(aq)}) from said hydrolysis reactor (3), and to supply said recovered aqueous solution of hydrogen halide (HX_{(aq)}) to the electrolysis unit (4).

With preference, the system further comprises a polyhalogenated alkane removal unit (13), configured to receive an effluent stream from said halogenation reactor (1) and configured to separate from said effluent stream (i) a polyhalogenated alkane stream, comprising polyhalogenated alkanes; and, (ii) a mono-halide stream, comprising methyl halide (MeX) and hydrogen halide (HX), and optionally comprising unreacted methane. For example, the hydrolysis reactor (3) is further fluidly connected to said polyhalogenated alkane removal unit (13) and further configured to receive a mono-halide stream from said polyhalogenated alkane removal unit (13), and wherein said hydrolysis reactor (3) is configured to react the mono-halide stream, with water and said organic base, under reaction conditions effective to produce an aqueous solution of hydrogen halide (HX_{(aq)}). Advantageously, the effluent recovery system (10, 15) is further configured for feeding said recovered effluent stream to said polyhalogenated alkane removal unit (13). Advantageously, the system further comprises a mono-halide transfer system (16), configured to recover a mono-halide stream from said polyhalogenated alkane removal unit (13), and to supply said mono-halide stream to the hydrolysis reactor (3).

For example, the system further comprises a water supply line (17) for supplying water to said hydrolysis reactor (3). With preference, the water supply line (17) is for supplying water and an organic base.

For example, the system further comprises a C₂₊ alkyl monohalide removal unit, configured to receive effluent stream from said halogenation reactor (1) and to remove C₂₊ alkyl monohalides from said effluent stream

As used herein, the term “fluidly connected” or “in fluid connection” are used herein as synonyms and intend to refer to a connection between two components (or units or devices or the like) of the system as given herein, so that through said connection a fluid can flow from one component (e.g., unit, device, reactor, etc) to the other. Preferably two fluidly connected components are connected so that an outlet of the first component is connected by a pipe, a conduit or a series of pipes or conduits, to an inlet of the second component. Other components, such as separators, dryers, condensers, and the like, may be fluidly connected between two fluidly connected components.

The terms “means for supplying” or “supply system” are used herein as synonyms and refer to suitable systems allowing the supply of a stream, or reaction product, or reactant, and includes for instance pipes, conduits, supply lines, inlet lines, connecting lines, and the like.

The term “means for recovering” or “recovery system” are used herein as synonyms and refer to suitable systems allowing the recovery of a stream or reaction product or reactant, and includes for instance pipes, recovery lines, outlet lines, connecting lines, and the like. “Means for recovering” or “recovery system” may comprise separation means.

The term “means for transferring” or “transfer system” are also used herein as synonyms and refer to suitable systems allowing the transfer (transport) of a stream or reaction product or reactant, e.g., from one unit or reactor to another unit or reactor, and includes for instance pipes, conduits, recovery lines, outlet lines, connecting lines, and the like.

A halogenation reactor (1) for use in a system of the disclosure, may be any type of reactor suitable for allowing a gaseous feed stream comprising methane to react with a halogen reactant under suitable conditions for yielding an effluent stream comprising methyl halide (MeX), and hydrogen halide (HX).

In certain embodiments of the present disclosure, polyhalogenated alkanes may be formed in step b) of the process. Such polyhalogenated alkanes can for instance comprise methyl dihalide (CH₂X₂). Therefore, a system according to the present disclosure may comprise in certain embodiments a polyhalogenated alkane removal unit, configured to receive effluent stream from said halogenation reactor (1) and to remove polyhalogenated alkanes from said effluent stream, thereby producing a polyhalogenated alkane stream and a mono-halide stream.

In certain preferred embodiments, a polyhalogenated alkane removal unit (13), for use in a system of the disclosure comprises or consists of a separation column.

Preferably, such polyhalogenated alkane removal unit is provided downstream of the halogenation reactor (1) and upstream of the hydrolysis reactor (3), and is connected, preferably fluidly connected, to the halogenation reactor (1) and to the hydrolysis reactor (3), e.g., by means of connecting lines (15, 16).

The polyhalogenated alkane removal unit may for instance comprises:
a separation system (13), such as a separation (e.g., distillation) column, configured to receive an effluent stream from said halogenation reactor (1) and to separate from said effluent stream a polyhalogenated alkanes stream;
a supply system (15), such as a supply line or pipe, configured for supplying an effluent stream from said halogenation reactor (1) to said separation column, and
a polyhalogenated alkane recovery system (14), such as an outlet line or pipe, configured for recovering a polyhalogenated alkanes stream from said separation system (13).

The polyhalogenated alkanes removal unit preferably also comprises means, such as a transfer line or pipe, for recovering a product stream (e.g., a product stream or a mono-halide stream comprising methyl halide and hydrogen halide), from which polyhalogenated alkanes as defined herein have been removed. Preferably such means are configured to transfer the recovered product stream to a downstream separation system, for further separation of the product stream, or the hydrolysis reactor (3).

In certain embodiments, the polyhalogenated alkanes removal unit is able to separate polyhalogenated alkanes from the effluent stream into a polyhalogenated methane stream and a polyhalogenated C₂₊ alkane stream. In certain embodiments, the polyhalogenated alkanes removal unit comprises a polyhalogenated methane outlet and a polyhalogenated C₂₊ alkanes outlet.

A hydrolysis reactor (3) for use in a system of the disclosure may be any type of reactor suitable for housing water and at least one catalyst which is preferably an organic base, as defined herein, and allowing a (gaseous) mono-halide stream to react with said water, and optionally at least one catalyst which is preferably an organic base under suitable conditions for producing an aqueous solution of hydrogen halide (HX_{(aq)}), and methanol (MeOH) and/or dimethyl ether (DME).

In certain embodiments, the hydrolysis reactor may comprise separation means for:
separating methanol and/or DME from the hydrolysis reactor; and/or
separating aqueous hydrogen halide solution, optionally comprising catalyst, from the hydrolysis reactor.

Examples of such separation means may be one or more distillation columns.

In certain embodiments, the hydrolysis reactor comprises means for retaining the catalyst, preferably the heterogeneous catalyst, inside the hydrolysis reactor. An example of such means is a filter on the reactor outlets, wherein the filter can retain solid catalyst or catalyst immobilised on solid support.

An electrolysis unit (4) for use in the present disclosure comprises at least one electrolysis cell and a power source for supplying current to said electrolysis cell. A particularly suitable electrolysis cell is a polymer electrolyte membrane cell (PEM) containing at least a proton-conductive membrane. One of the largest advantages to PEM electrolysis is its ability to operate at high current densities. This can result in reduced operational costs. The polymer electrolyte allows a PEM electrolytic cell to operate with a very thin membrane (˜100-200 μm) while still allowing high pressures, resulting in low ohmic losses, primarily caused by the conduction of protons across the membrane (0.1 S/cm) and a compressed hydrogen output. The polymer electrolyte membrane, due to its solid structure, advantageously gives very high product gas purity.

In some embodiments, the electrolysis unit may comprise means for returning catalyst (which may have entered the electrolysis unit via the aqueous solution of hydrogen halide (HX_{(aq)}) to the hydrolysis reactor.

A system according to the present disclosure may also comprise
a feed stream supply system (5) for supplying a gaseous feed stream comprising methane to said halogenation reactor (1); and
a halogen supply system (6) for supplying a halogen reactant to said halogenation reactor (1).

Such systems for supplying reactants to the halogenation reactor may include inlet lines (conduits), optionally provided with controlling means for controlling flow rate of the reactant streams to the reaction zone. In preferred embodiments of the disclosure separate means (separate inlet lines or conduits) may be provided for each of the reactants in the process, i.e., separately for the gaseous feed stream and for the halogen reactant.

A system according to the present disclosure may also comprise an effluent recovery system (10), e.g., a conduit or pipe, configured to recover an effluent stream from said halogenation reactor (1), and adapt to feed said recovered effluent stream to the downstream polyhalogenated alkane removal unit (13) or to the hydrolysis reactor (3).

A system according to the present disclosure may also comprise:
a hydrogen halide transfer system (12), configured to recover an aqueous solution of hydrogen halide (HX_{(aq)}) from said hydrolysis reactor (3), and to supply said recovered hydrogen halide stream to the electrolysis unit (4); and
a mono-halide transfer system (11), configured to recover a mono-halide stream from polyhalogenated alkane removal unit (13), and to supply said recovered mono-halide stream to the hydrolysis reactor (3).

Such systems for supplying or recovering reactants to and from the separation system, may include inlet lines (conduits, pipes, lines), optionally provided with controlling means, e.g., for controlling flow rate of the reactant streams to the reaction zone.

A system according to the present disclosure may also comprise a water supply line (17), for supplying water, preferably water and catalyst, to the hydrolysis reactor. The water supply line is for instance an inlet line, for supplying water and optionally catalyst, to said separation zone.

A system in accordance with the present disclosure may also comprise systems for separately recovering a gaseous hydrogen stream and a stream comprising halogen reactant from the electrolysis unit. Such systems comprise in particular,
a hydrogen recovery system (8), configured to recover a gaseous hydrogen stream from said electrolysis unit (4);
a halogen recovery system (9), configured to recover halogen reactant from said electrolysis unit (4);

Such systems for supplying or recovering reactants to and from the separation system, may include inlet lines (conduits, pipes, lines), optionally provided with controlling means, e.g., for controlling flow rate of the reactant streams to the reaction zone.

Optionally, the system of the disclosure may be provided with means (20) (e.g., a transfer line or conduit) for returning the stream of halogen reactant recovered from said electrolysis unit, or least a part thereof, to the halogenation reactor. This allows for the re-use of halogen reactant in the system.

In certain preferred embodiments, the means (20) for returning the stream of halogen reactant, or a part thereof, recovered from said electrolysis unit (4) to said halogenation reactor (1) comprise one or more connecting lines (connecting pipes).

In certain embodiments, said one or more connecting lines for returning the stream of halogen reactant to said halogenation reactor may be provided with one of more dryers (e.g., molecular sieves), suitable for drying the stream of halogen reactant prior to feeding said stream to said halogenation reactor.

In certain embodiments, said one or more connecting lines for returning the stream of halogen reactant to said halogenation reactor may be provided with one of more purification means, such as e.g., one or more condensers, suitable for purifying the stream of halogen reactant prior to feeding said stream to said halogenation reactor.

Furthermore, the system of the disclosure comprises a methanol and/or dimethyl ether (DME) recovery system (7), configured to recover a stream comprising methanol and/or dimethyl ether (DME) from said hydrolysis reactor (3).

In certain embodiments of the present disclosure, C₂₊ alkyl monohalides, as defined herein, may be formed in step b) of the process. Therefore, in certain embodiments a system according to the present disclosure may also comprise a C₂₊ alkyl monohalide removal unit, configured to receive effluent stream from said halogenation reactor (1) and to remove C₂₊ alkyl monohalides from said effluent stream.

In certain embodiments, such C₂₊ alkyl monohalide removal unit is provided downstream of the halogenation reactor (1) and upstream of the hydrolysis reactor (3), and is connected, preferably fluidly connected, to the halogenation reactor (1) and to the hydrolysis reactor (3), e.g., by means of connecting lines.

In certain embodiments, a C₂₊ alkyl monohalide removal unit may for instance comprises
a separation system, such as a separation (e.g., distillation) column, configured to receive an effluent stream from said halogenation reactor (1) and to separate from said effluent stream a C₂₊ alkyl monohalide stream;
a supply system, such as a supply line or pipe, configured for supplying an effluent stream from said halogenation reactor (1) to said separation system, and
a C₂₊ alkyl monohalide recovery system, such as an outlet line or pipe, configured for recovering a C₂₊ alkyl monohalide stream from said C₂₊ alkyl separation system.

A C₂₊ alkyl monohalide removal unit preferably also comprises means, such as a transfer line or pipe, for recovering a product stream (e.g., a mono-halide stream as defined herein) from said C₂₊ alkyl monohalide removal. Preferably such means are configured to transfer the recovered product stream to the hydrolysis reactor (3).

In certain embodiments of the present disclosure, unreacted methane may remain present in the effluent stream obtained in step b) of the process. In certain embodiments of the present disclosure, unreacted methane may remain present in the mono-halide stream obtained in step c) of the process. In certain embodiments of the present disclosure, unreacted methane may remain present in the stream of methanol and/or dimethyl ether obtained in step d) of the present process. Remaining unreacted methane may be removed from said respected stream, e.g., by distillation. Therefore, the present system further provides means, such as distillation columns or the like, for removing, preferably distilling, unreacted methane, from above mentioned streams. In a particularly preferred embodiment of the present disclosure, a system is provided in which a distillation column is provided in fluid connection with the methanol and/or dimethyl ether recovery system (7).

The following examples serve to merely illustrate the disclosure and should not be construed as limiting its scope in any way. While the disclosure has been shown in only some of its forms, it should be apparent to those skilled in the art that it is not so limited, but is susceptible to various changes and modifications without departing from the scope of the disclosure.

### EXAMPLES

### Example 1

The following example demonstrates an embodiment of a process according to the disclosure, and a system for carrying out such embodiment according to the disclosure, with reference to FIG. 1.

The system depicted in FIG. 1 comprises a halogenation reactor (1), a hydrolysis reactor (3) and an electrolytic unit (4).

According to this embodiment, a gaseous feed stream comprising methane is introduced into the halogenation reactor (1) via a feed stream supply line (5). A halogen reactant (e.g. Br₂) is also introduced into this halogenation reactor (1), through a separate halogen reactant supply line (6). The gaseous feed stream comprising methane reacts with the halogen reactant (X₂) in the halogenation reactor, yielding an effluent stream comprising methyl halide (MeX) and hydrogen halide (HX). Optionally unreacted methane remains present in this effluent stream.

The obtained effluent stream exits the halogenation reactor (1) through a suitable recovery line (10) and is fed via said line into the hydrolysis reactor (3). In the hydrolysis reactor (3) the effluent stream is reacted with a water and optionally at least one catalyst, whereby on the one hand an aqueous solution of hydrogen halide (HX_{(aq)}) is formed and on the other hand methanol (MeOH) and/or dimethyl ether (DME) is formed. A stream comprising methanol and/or dimethyl ether (DME) can be recovered from the hydrolysis reactor (3) through a methanol and/or dimethyl ether (DME) recovery line (7). The water and optionally catalyst, may be added to the hydrolysis reactor (3) via a water supply line (17).

The in the hydrolysis reactor (3) formed aqueous solution of hydrogen halide (HX_{(aq)}), is fed via a suitable connecting line (12), to a electrolysis unit (4). In the electrolysis unit (4), the aqueous solution of hydrogen halide (HX_{(aq)}), issued from the hydrolysis reactor (3), is decomposed into stream of hydrogen gas (H₂), which is removed from the electrolysis unit via outlet line (8); and a stream of halogen reactant (X₂), which is removed from the electrolysis unit via outlet line (9).

### Example 2

The following example demonstrates another embodiment of a process according to the disclosure, and a system for carrying out such embodiment according to the disclosure, with reference to FIG. 2.

The system depicted in FIG. 1 comprises a halogenation reactor (1), a polyhalogenated alkane removal unit (13), a hydrolysis reactor (3), and an electrolytic unit (4).

A gaseous feed stream comprising methane containing about 100 mol. % (CH₄) was introduced into a halogenation reactor (1) via feed stream supply line (5) at a flow rate of about 1604 kg/hr. The halogen reactant (X₂), in this case bromine (Br₂), was introduced to the halogenation reactor (1) through the halogen reactant supply line (6) at a rate of about 5274 kg/hr. The halogenation reactor (1) was maintained at a temperature of about 300-600° C. and a pressure from 5 to 30 bar.

An effluent stream comprising unreacted methane (CH₄), hydrogen halide (HX), methyl halide (MeX), and polyhalogenated alkanes (e.g. CH₂X₂) was recovered from the halogenation reactor (1), via outlet (15) and fed to a polyhalogenated alkane removal unit comprising a separation column (13). This separation column separates the polyhalogenated alkane (e.g. CH₂X₂), e.g. CH₂Br₂, from the effluent stream and removes the polyhalogenated alkane (e.g. CH₂X₂) from the system via a polyhalogenated alkane outlet line (14) at rate of around 860 kg/hr.

The residual gaseous stream, i.e. the mono-halide stream, which was substantially free of polyhalogenated alkane (e.g. CH₂X₂), was recovered from the polyhalogenated alkane removal unit (13), and was then fed, via a connecting line (16), into to the hydrolysis reactor (3). In the hydrolysis reactor (3) the effluent stream was reacted with water and at least one catalyst, i.e. a 15 vol % pyridine aqueous solution, at a temperature of 80° C. This resulted, on the one hand in the formation of an aqueous solution of hydrogen halide (HX_{(aq)}) and on the other hand in the formation of methanol (MeOH) at a rate of 312 kg/hr and dimethyl ether (DME) at a rate of 250 kg/hr. The stream comprising methanol and dimethyl ether (DME) was be recovered from the hydrolysis reactor (3) through a methanol recovery line (7). The 15 vol % pyridine aqueous solution is added to the hydrolysis reactor (3) via a water supply line (17) at a rate of 1715 kg/hr.

Methanol, dimethyl ether and optionally unreacted methane recovered from the recovery line (7) can be separated by techniques known in the art, e.g. distillation with a distillation column. The aqueous solution of hydrogen halide formed in the hydrolysis reactor (3) is fed via a suitable connecting line (12), to a electrolysis unit (4). In the electrolysis unit (4), the aqueous solution of hydrogen halide (HX_{(aq)}), issued from the hydrolysis reactor (3), is decomposed into stream of hydrogen gas (H₂), which is removed from the electrolysis unit via outlet line (8); and a stream of halogen reactant (X₂), which is removed from the electrolysis unit via outlet line (9). The produced halogen reactant (X₂) may also be recovered through an outlet line (9), optionally dried, and returned to the halogenation reactor (1) for re-use.

### Example 3

This example illustrates a halogenation step as may be applied in a process of the disclosure.

In this example, the experimental setup comprised a quartz reactor and a glass syringe for liquid bromine supply (Sigma Aldrich). A methane (5.0, Praxair) supply with Bronkhorst mass-flow controller was created.

The feed rate was maintained to have a residence time for reactants in the heated zone of the reactor of around 0.01 s and the molar ratio of CH₄/Br₂ of about 3/1. The heated zone of the reactor was kept at a temperature of about 370° C. and pressure of about 3 bar.

The effluent of the reactor was then first passed through a cold trap (10° C.) to condense CH₂Br₂, produced during the reaction, and trace amounts of unreacted bromine.

In this experiment, the effluent of the reactor was passed through an aqueous caustic bubbler to remove major amount of HBr prior to sending the remaining stream for analysis to an Agilent GC with SilicaPlot column and FID detector.

The conversion of methane into methyl bromide, with side production of CH₂Br₂, was calculated on the basis of GC data, using the following formula:

$$x ( CH 4 ) = \frac{n ( CH {4}_{f e e d} ) - n ( CH {4}_{effluent} )}{n ( CH {4}_{f e e d} )}$$

$$s ( CH 3 Br ) = \frac{n ( CH 3 {Br}_{effluent} )}{n ( CH {4}_{f e e d} ) - n ( CH {4}_{effluent} )}$$

$$s ( CH 2 Br 2 ) = \frac{n ( CH 2 Br {2}_{effluent} )}{n ( CH {4}_{f e e d} ) - n ( CH {4}_{effluent} )}$$

x(CH₄)=29%, s(CH₃Br)=89%, s(CH₂Br₂)=9%;
wherein n=molar quantity, x=conversion and s=selectivity

### Example 4

The following example illustrates a hydrolysis reaction step as may be applied in a process of the disclosure.

In this example, the experimental setup comprised a batch reactor (300 ml) connected to a GC was used. The reactor was charged with 200 ml deionized water and 30 ml pyridine dissolved in said water. Methyl bromide (>99.9%, Mebrom) was fed through a piston pump.

The temperature of the reactor was kept at 80° C. during the entire experiment and vigorous mixing was kept with a magnetic stirrer. Flowrates of CH₃Br of 5 g/h and of N₂ of 70 mL/min were used during the experiment. The conversion of CH₃Br into methanol and dimethyl ether appeared to be stable during 3 h of the run.

The conversion of methyl bromide into methanol was calculated on the basis of GC data, using the following formula:

$$x ( CH 3 Br ) = \frac{n ( CH 3 {Br}_{f e e d} ) - n ( CH 3 {Br}_{effluent} )}{n ( CH 3 {Br}_{f e e d} )}$$

$$s ( CH 3 OH ) = \frac{n ( CH 3 {OH}_{effluent} )}{n ( CH 3 {Br}_{f e e d} ) - n ( CH 3 {Br}_{effluent} )}$$

$$s ( DM E ) = \frac{2 * n ( D M {E}_{e f f l u e n t} )}{n ( CH 3 {Br}_{f e e d} ) - n ( CH 3 {Br}_{e f f l u e n t} )}$$

With x(CH₃Br)=14%, s(CH₃OH)=32% and s(DME)=68%;
wherein n=molar quantity, x=conversion and s=selectivity

1-32. (canceled)

33. A process for producing methanol (MeOH), dimethyl ether (DME), and hydrogen (H₂) from methane, comprising the steps of:
a) providing a gaseous feed stream comprising methane;
b) reacting said gaseous feed stream with at least one halogen reactant (X₂), under reaction conditions effective to produce an effluent stream comprising methyl halide (MeX), hydrogen halide (HX), polyhalogenated alkanes, and optionally unreacted methane;
c) recovering an effluent stream comprising methyl halide (MeX), hydrogen halide (HX), optionally polyhalogenated alkanes, and optionally unreacted methane;
d) reacting the effluent stream obtained in step c) with water and an organic base, under reaction conditions effective to produce:
an aqueous solution of hydrogen halide (HX_{(aq)}), and
a methanol stream comprising methanol (MeOH) and dimethyl ether (DME) and optionally unreacted methane, and,
e) decomposing by means of electrolysis said aqueous solution of hydrogen halide (HX(aq)) under conditions effective to produce a gaseous hydrogen (H₂) stream and a stream comprising halogen reactant (X₂);
wherein the process comprises the step of separating said polyhalogenated alkanes formed in step (b) from said effluent stream.

34. The process according to claim 33, wherein the step of separating said polyhalogenated alkanes is carried out prior to reacting the effluent stream with water and an organic base.

35. The process according to claim 33, wherein said halogen reactant is selected form the group consisting of bromine (Br₂), chlorine (Cl₂), fluorine (F₂), iodine (I₂), and astatine (At₂).

36. The process according to claim 33, wherein an organic base is an N-containing polymer.

37. The process according to claim 36, wherein the N-containing polymer is a polymer comprising at least one amino group, pyridine ring, pyridazine ring, pyrimidine ring, pyrazine ring, triazine ring, pyrazole ring, imidazole ring, oxazole ring, isoxazole ring, thiazole ring, isothiazole ring, triazole ring, oxadiazole ring, or any combinations thereof.

38. The process according to claim 36, wherein the N-containing polymer is a polymer comprising at least one pyridine ring.

39. The process according to claim 33, wherein said polyhalogenated alkanes comprise polyhalogenated methane and optionally polyhalogenated C₂₊ alkanes.

40. The process according to claim 33, wherein C₂₊ alkyl monohalides are formed in step b).

41. The process according to claim 33, further comprising the step of returning the halogen reactant (X₂) obtained in step e), to step b) of the process.

42. The process according to claim 33, wherein methanol stream comprising methanol (MeOH), and dimethyl ether (DME), obtained in step d2) is separated from the aqueous solution of hydrogen halide by means of distillation.

43. The process according to claim 42, wherein the methanol stream comprising methanol (MeOH) and dimethyl ether (DME) obtained in step d2) comprises at least 75.0 mol. %, of methanol and dimethyl ether in combination, based on the total amount of the methanol stream.

44. The process according to claim 42, wherein the methanol stream comprising methanol (MeOH) and dimethyl ether (DME) obtained in step d2) comprises at least 95.0 mol. % of methanol and dimethyl ether in combination based on the total amount of the methanol stream.

45. The process according to claim 33, wherein the gaseous hydrogen (H₂) stream generated in step e) comprises at least 90.0 mol. % of hydrogen.

46. The process according to claim 33, wherein the gaseous hydrogen (H₂) stream generated in step e) comprises at least 99.9 mol. % of hydrogen.

47. System for producing methanol (MeOH), dimethyl ether (DME) and hydrogen (H₂) from methane, comprising:
a halogenation reactor (1), configured to react a gaseous feed stream comprising methane with at least one halogen reactant (X₂) into an effluent stream comprising methyl halide (MeX), and hydrogen halide (HX), and optionally unreacted methane; and,
a hydrolysis reactor (3), which is fluidly connected to said halogenation reactor (1) and configured to receive an effluent stream from said halogenation reactor (1); and, wherein said hydrolysis reactor (3) is configured to react the effluent stream with water and an organic base, under reaction conditions effective to produce a stream comprising methanol (MeOH) and dimethyl ether (DME) and optionally unreacted methane; and
an electrolysis unit (4) comprising at least one electrolysis cell and a power source for supplying current to said electrolysis cell, which is fluidly connected to said hydrolysis reactor (3), and configured to receive an aqueous solution of hydrogen halide (HX_{(aq)}) and to decompose said aqueous solution of hydrogen halide (HX_{(aq)}) into a gaseous hydrogen (H₂) stream and a stream comprising halogen reactant (X₂);
a feed stream supply system (5) for supplying a gaseous feed stream comprising methane to said halogenation reactor (1);
a halogen supply system (6) for supplying a halogen reactant (X₂) to said halogenation reactor (1);
an effluent recovery system (10, 15), configured to recover an effluent stream from said halogenation reactor (1), and for feeding said recovered effluent stream to said hydrolysis reactor (3);
a methanol recovery system (7), configured to recover a stream comprising methanol (MeOH), and dimethyl ether (DME) and optionally unreacted methane from said hydrolysis reactor (3);
a hydrogen recovery system (8), configured to recover a gaseous hydrogen (H₂) stream from said electrolysis unit (4);
a halogen recovery system (9), configured to recover halogen reactant (X₂) from said electrolysis unit (4);
a hydrogen halide transfer system (12), configured to recover an aqueous solution of hydrogen halide (HX_{(aq)}) from said hydrolysis reactor (3), and to supply said recovered aqueous solution of hydrogen halide (HX_{(aq)}) to the electrolysis unit (4);
a polyhalogenated alkane removal unit (13), configured to receive an effluent stream from said halogenation reactor (1) and configured to separate from said effluent stream (i) a polyhalogenated alkane stream, comprising polyhalogenated alkanes; and, (ii) a mono-halide stream, comprising methyl halide (MeX) and hydrogen halide (HX), and optionally comprising unreacted methane.

48. System according to claim 47 wherein the hydrolysis reactor (3) is further fluidly connected to said polyhalogenated alkane removal unit (13) and further configured to receive a mono-halide stream from said polyhalogenated alkane removal unit (13), and wherein said hydrolysis reactor (3) is configured to react the mono-halide stream, with water and said organic base, under reaction conditions effective to produce an aqueous solution of hydrogen halide (HX_{(aq)}).

49. System according to claim 47, wherein the effluent recovery system (10, 15) is further configured for feeding said recovered effluent stream to said polyhalogenated alkane removal unit (13).

50. System according to claim 47, wherein the system further comprises a mono-halide transfer system (16), configured to recover a mono-halide stream from said polyhalogenated alkane removal unit (13), and to supply said mono-halide stream to the hydrolysis reactor (3).

51. System according to claim 47, wherein the system further comprises a water supply line (17) for supplying water to said hydrolysis reactor (3).

52. System according to claim 47, wherein the system further comprises a C₂₊ alkyl monohalide removal unit, configured to receive effluent stream from said halogenation reactor (1) and to remove C₂₊ alkyl monohalides from said effluent stream.
